(19) United States
(12) Patent Application Publication
(10) Pub. No.: US 2026/0207572 A1
(43) Pub. Date: Jul. 23, 2026

(54) METHODS AND COMPOSITIONS FOR TREATMENT OF SUBJECTS WITH HEPATIC IMPAIRMENTS

(71) Applicant: ELEVAR THERAPEUTICS, INC., Fort Lee, NJ (US)

(72) Inventor: Seong Hoon Jang, Gaithersburg, MD (US)

(21) Appl. No.: 19/139,670

(22) PCT Filed: Dec. 14, 2023
(86) PCT No.: PCT/US2023/084119

§ 371 Date: Jun. 16, 2025

Related U.S. Application Data
(60) Provisional application No. 63/433,162, filed on Dec. 16, 2022.

(51) Int. Cl. A61K 31/444

(52) U.S. Cl. CPC A61K 31/444

(57) ABSTRACT

Provided herein are methods and rivoceranib compositions for the treatment of diseases, e.g., cancer, by administering to a subject having mild or moderate hepatic impairment a therapeutically effective amount of rivoceranib.

### FIELD OF THE DISCLOSURE

The present disclosure relates to methods and compositions for treating cancer in subjects with hepatic impairment.

### BACKGROUND OF THE DISCLOSURE

Tumor angiogenesis plays a critical role in malignant tumor growth and metastasis. When tumors grow beyond 1 mm³, angiogenesis, or generation of vascular arborizations by budding from existing vessels, is necessary to provide enough blood for the survival of tumor cells. The growth speed and tendency of metastasis of tumors are associated with the level of neovascularization factors and the quantity of nascent microvessels. Since the hypothesis of “anti-angiogenesis therapy” was put forward by Folkman in early 1970s, people have made considerable progress in this field, and inhibiting angiogenesis of tumors has been universally accepted as a promising anticancer strategy.

Rivoceranib (also known as YN968Dl, developed in China as apatinib and marketed as Aitan®) is an orally administered small molecule tyrosine kinase inhibitor with selectivity towards the VEGFR-2/kinase insert domain receptor. In tumor tissues, VEGF binds to VEGFR, stimulates VEGFR-mediated downstream signaling transduction, and ultimately leads to tumor angiogenesis. The classical VEGFRs in mammals include VEGFR1 Fms related receptor tyrosine kinase 1 (FLT1), VEGFR2 kinase insert domain receptor (KDR) and VEGFR3 (FLT4). Compared with the other 2 VEGFRs, VEGFR2 has higher affinity and kinase activity and is more important for direct regulation of angiogenesis, mitogenic signaling, and permeability-enhancing effects. Most VEGFRs are expressed by many tumor types, and their expression levels correlate with poor clinical outcomes. Rivoceranib selectively binds to and inhibits VEGFR2, which is believed to be principally responsible for inhibition of VEGF-stimulated endothelial cell migration and proliferation and decreases in tumor microvascular density. Rivoceranib has received approval in China, for treatment of advanced gastric cancer, and has received orphan medicinal product designation for the treatment of gastric cancer from Europe, the FDA, and the MFDS in South Korea.

Hepatic impairment is a condition wherein normal functioning of the liver is reduced. Hepatic impairment can be acute, with rapid onset, or chronic. Chronic hepatic impairment, or cirrhosis, can occur from many causes, such as excessive consumption of alcohol, hepatitis, autoimmune disease, heredity, or metabolism, or can be idiopathic. Liver damage is generally irreversible, and treatment consists of prevention of progression and treatment of symptoms. Hepatic impairment can exhibit no significant symptoms, or may be characterized by such symptoms as reduced ability for the blood to clot (coagulopathy) and brain dysfunction (encephalopathy), fluid retention in the abdominal cavity, increased infection risk, hypogonadism, change in liver size, jaundice, and increased sensitivity to medication.

The changes in pharmacokinetic parameters such as AUC, Cₘₐₓ, t_{1/2} of a drug, and/or its metabolites, in patients with hepatic impairment can lead to many problems, including a need for adjusting dose, complications for physicians in prescribing, need for liver function tests, lack of availability of correct doses, lack of availability of certain medications to those with hepatic impairment, and overdosing.

### SUMMARY OF THE DISCLOSURE

The disclosure is based, at least in part, on the discovery that if you treat cancer in subjects having mild or moderate hepatic impairment using the same therapeutically effective amounts of rivoceranib or a pharmaceutically acceptable salt thereof as used in subjects with normal hepatic function, the cancer in the subject with mild to moderate hepatic impairment improves with acceptable adverse effects. The disclosure demonstrates that subjects with mild or moderate hepatic impairment do not have a statistically significant difference in exposure of rivoceranib compared to exposure in a subject with normal hepatic function who is administered the same amount of rivoceranib.

In a first aspect, the present disclosure provides methods of treating cancer in a subject having mild or moderate hepatic impairment. The methods include administering rivoceranib or a pharmaceutically acceptable salt thereof to the subject having mild or moderate hepatic impairment, wherein rivoceranib or a pharmaceutically acceptable salt thereof is administered in a total daily amount in a range of about 100 mg to about 700 mg.

Also provided herein are methods of treating cancer in a subject having mild or moderate hepatic impairment. The methods include assessing hepatic function of the subject; determining that the subject has mild or moderate hepatic impairment; and administering to the subject with mild or moderate hepatic impairment a therapeutically effective amount of rivoceranib or a pharmaceutically acceptable salt thereof that is equal to a therapeutically effective amount for a subject with normal hepatic function, wherein the therapeutically effective amount of rivoceranib or a pharmaceutically acceptable salt thereof is administered in a total daily amount of about 100 mg to about 700 mg. The hepatic function can be assessed by a Child-Pugh Scale as described herein.

In another aspect, the disclosure provides compositions comprising or consisting of rivoceranib or a pharmaceutically acceptable salt thereof for use in the treatment of cancer in a subject having mild or moderate hepatic impairment, wherein the rivoceranib or a pharmaceutically acceptable salt thereof is administered to the subject in a total daily in a range of about 100 mg to about 700 mg.

Also provided herein are dosing regimens for rivoceranib or a pharmaceutically acceptable salt thereof in treatment of cancer in a subject having a mild or moderate hepatic impairment, the dosing regimens include administering a total daily amount of rivoceranib or a pharmaceutically acceptable salt thereof in a range of about 100 mg to about 700 mg for at least one cycle, wherein each cycle is at least 10 days, e.g., at least 28 days.

In certain embodiments of all aspects disclosed herein, the pharmaceutically acceptable salt of rivoceranib is rivoceranib mesylate salt, and the total daily amount of rivoceranib or a pharmaceutically acceptable salt thereof is about 100 mg, about 200 mg, about 300 mg, about 400 mg, about 500 mg, about 600 mg, or about 700 mg, e.g., at least about 200 mg.

In various embodiments, the rivoceranib or a pharmaceutically acceptable salt thereof is administered orally, e.g., in tablet form, e.g., once or twice daily.

In these methods and compositions for use in the treatment of cancer, the subject may have mild or moderate hepatic impairment, e.g., the subject has a Child-Pugh Score of 5-6 or 7-9. A subject with severe hepatic impairment would have a Child-Pugh score of 10-15, e.g., 10 or more. Subjects with severe hepatic impairment may be advised not to take rivoceranib, or to take a low dose of rivoceranib, e.g., about 100, 125, 150, 175, or up to about 200 mg.

The methods and compositions can be used to treat a wide variety of cancers, e.g., as listed herein. For example, the cancer can be a liver cancer, e.g., selected from hepatocellular carcinoma, hepatoma, cholangiocarcinoma, hepatoblastoma, hepatic carcinoma, hepatic angiosarcoma, and/or metastatic liver cancer. For example, the cancer can be hepatocellular carcinoma, gastric caner, or adenoid cystic carcinoma.

In the new methods and compositions, the rivoceranib or a pharmaceutically acceptable salt thereof can be administered for at least 10, 15, 21, or 28 days or more. In some embodiments, the rivoceranib or a pharmaceutically acceptable salt thereof can be administered for at least 28 days.

In certain embodiments, the subject with mild or moderate hepatic impairment does not have a statistically significant difference in exposure of rivoceranib, compared to exposure in a subject with normal hepatic function who is administered the same amount of rivoceranib. In these methods, the exposure can be measured by Cmax or AUC0-∞.

In the new methods, administration of the rivoceranib or a pharmaceutically acceptable salt thereof is discontinued when the patient no longer has cancer or, optionally, when the subject has a Child-Pugh score of 10 or higher. In some embodiments, if the subject is determined to have severe hepatic impairment, e.g., a Child-Pugh score of 10 or higher, rivoceranib or a pharmaceutically acceptable salt thereof is not administered to that subject.

The new methods and compositions for use in the treatment of cancer provide significant advantages for patients with hepatic impairment, because given the information and data presented in the present disclosure, these patients can now be treated with the same or equal doses of rivoceranib or pharmaceutically acceptable salts thereof as patient with normal hepatic function.

Hepatic impairment can cause alterations in drug disposition and pharmacokinetics (PK). Such alterations can reduce the clearance of drugs eliminated by hepatic metabolism or biliary excretion and affect plasma protein binding, which in turn could influence the processes of distribution and elimination of drugs in a subject. Based on prior studies of rivoceranib, hepatic impairment was expected to impact the PK of rivoceranib, perhaps significantly, and to change the unbound plasma concentrations of rivoceranib based on data obtained through previous clinical studies. Therefore, there is a need for methods of treating cancer in a subject with hepatic impairments comprising administering rivoceranib or a pharmaceutically acceptable salt thereof to the subject in a correct dosage. The present disclosure fulfills these and other needs, as evident in reference to the following disclosure.

Advantageously provided herein are methods and compositions for treating cancer in subjects with mild or moderate hepatic impairment. The present disclosure demonstrates that the disclosed methods can be used in subjects having mild or moderate hepatic impairment with zero to tolerable adverse events.

As used herein, the term “subject” can be a vertebrate, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the subject is a mammal. In one aspect, the subject is a human. In one aspect, the subject is a patient. A patient refers to a subject afflicted with a disease or disorder. The term “patient” includes human and veterinary subjects. In some aspects of the disclosed methods, the subject has been diagnosed with a need for treatment of a disorder of uncontrolled cellular proliferation, such as cancer, e.g., hepatocellular carcinoma, gastric cancer, or adenoid cystic carcinoma.

As used herein, the term “treat,” “treating,” or “treatment” refers to the medical management of a subject with the intent to cure, ameliorate, or stabilize a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e.g., a human), and includes: (i) inhibiting the disease, i.e., arresting its development; or (ii) relieving the disease, i.e., causing regression of the disease.

A “therapeutically effective amount” when used in connection with a pharmaceutical composition described herein is an amount of one or more pharmaceutically active agent(s) sufficient to produce a therapeutic result in a subject in need thereof.

As used herein, the term “diagnosed or diagnosing” means having been subjected to a physical examination by a person of skill, for example, a physician, and found to have a condition that can be diagnosed or treated by the compounds, compositions, or methods disclosed herein. For example, “diagnosed with a disorder of uncontrolled cellular proliferation” means having been subjected to a physical examination by a person of skill, for example, a physician, and found to have a condition that can be diagnosed or treated by a compound or composition that can inhibit uncontrolled cellular proliferation. Such a diagnosis can be in reference to a disorder, such as a disorder of uncontrolled cellular proliferation, cancer and the like, as discussed herein.

As used herein, the term “assessing” or “assessed” refers to a form of measurement, including determining if a disease or disorder is present or not, as well as, in some instances, determining the amount of something present and/or determining the change of the amount of something present over time. In some embodiments, assessing a subject can lead to a subject being identified as needing treatment for a disorder. As used herein, the phrase “identified to be in need of treatment for a disorder,” or the like, refers to selection of a subject based upon need for treatment of the disorder. For example, a subject can be identified as having a need for treatment of a disorder based upon an earlier diagnosis by a person of skill and thereafter subjected to treatment for the disorder. It is contemplated that the identification can, in one aspect, be performed by a person different from the person making the diagnosis. It is also contemplated, in a further aspect, that the administration can be performed by one who subsequently performed the administration.

As used herein, “hepatic impairment” means hepatocellular (liver) dysfunction.

As used herein, “Child-Pugh Score” is a score based on five clinical measures of hepatic impairment, including levels of total bilirubin, serum albumin, PT INR, ascites, and hepatic encephalopathy. Each measure is given a ranking of 1, 2, or 3, and the sum of the five rankings is the Child-Pugh Score. The Child-Pugh Score can be used to classify hepatic impairment by placing subjects in a Child-Pugh Group. As used herein, “mild hepatic impairment” refers to a ranking of level of hepatic impairment based on a Child-Pugh Score of 5-6. As used herein, “moderate hepatic impairment” refers to a ranking of level of hepatic impairment based on a Child-Pugh Score of 7-9. As used herein, “severe hepatic impairment” refers to a ranking of level of hepatic impairment based on a Child-Pugh Score of 10-15.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration, intraurethral administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition.

As used herein, the term “pharmaceutically acceptable carrier” relates to pharmaceutically acceptable, nontoxic carriers or diluents, which are defined as vehicles commonly used to formulate pharmaceutical compositions for animal or human administration. Such carriers may include, however not limited to, buffering agents, solubilizing agents, stabilizing agents or taste additives.

As used herein, “AUC” refers to the area under the curve, or the integral, of the plasma concentration of an active pharmaceutical ingredient or metabolite over time following a dosing event.

As used herein “AUC₀₋ₜ” is the integral under the plasma concentration curve from time 0 (dosing) to time “t”.

As used herein, “AUC_{0-∞}” is the AUC from time 0 (dosing) to time infinity. Unless otherwise stated, AUC refers to AUC_{0-∞}. Often a drug is packaged in a salt form, for example rivoceranib mesylate salt, and the dosage form strength refers to the mass of this salt form or the equivalent mass of the corresponding free base, rivoceranib.

As used herein, Cₘₐₓ is a pharmacokinetic parameter denoting the maximum observed blood plasma concentration following delivery of an active pharmaceutical ingredient. Cₘₐₓ occurs at the time of maximum plasma concentration, tₘₐₓ.

As used herein, “tₘₐₓ” is a pharmacokinetic parameter denoting the time to maximum blood plasma concentration following delivery of an active pharmaceutical ingredient.

As used herein, “t_{1/2}” or “plasma half-life” or “elimination half-life” or the like is a pharmacokinetic parameter denoting the apparent plasma terminal phase half-life, i.e., the time, after absorption and distribution of a drug is complete, for the plasma concentration to fall by half.

In this application, the use of the singular includes the plural unless specifically stated otherwise. It must be noted that, as used in the specification, the singular forms “a,” “an,” and “the” include plural referents unless the context clearly dictates otherwise. In this application, the use of “or” means “and/or” unless stated otherwise. Furthermore, use of the term “including” as well as other forms, such as “include,” “includes,” and “included,” are not limiting.

As used herein, ranges and amounts can be expressed as “about” a particular value or range. About also includes the exact amount. Hence “about 5 μl” means “about 5 μl” and also “5 μl.” Generally, the term “about” includes an amount that would be expected to be within experimental error. Unless specifically stated or obvious from context, as used herein, the term “about” in reference to a number or range of numbers is understood to mean the stated number and numbers +/−10% thereof, or 10% below the lower listed limit and 10% above the higher listed limit for the values listed for a range.

Unless defined otherwise, all technical and scientific terms used herein have the same meaning as is commonly understood by one of skill in the art to which the claimed subject matter belongs. It is to be understood that the detailed description provides examples and explanations only, which are not restrictive of any subject matter claimed. All publications, patent applications, patents, and other references mentioned herein are incorporated by reference in their entirety. In case of conflict, the present specification, including definitions, will control. In addition, the materials, methods, and examples are illustrative only and not intended to be limiting.

Although methods and materials similar or equivalent to those described herein can be used in the practice or testing of the present invention, suitable methods and materials are described below. Although various features of the disclosure may be described in the context of a single embodiment, the features may also be provided separately or in any suitable combination. Conversely, although the disclosure may be described herein in the context of separate embodiments for clarity, the disclosure may also be implemented in a single embodiment.

Reference in the specification to “some embodiments,” “an embodiment,” “one embodiment,” or “other embodiments” means that a particular feature, structure, or characteristic described in connection with the embodiments is included in at least some embodiments, but not necessarily all embodiments, of the disclosures.

The section headings used herein are for organizational purposes only and are not to be construed as limiting the subject matter described.

Other features and advantages of the methods and compositions of the disclosure will be apparent from the following detailed description, and from the claims.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings.

FIG. 1 is a linear scale graph of the arithmetic mean plasma concentration-time profiles of rivoceranib following administration of rivoceranib or a pharmaceutically acceptable salt thereof in subjects with normal hepatic function, mild hepatic impairment, and moderate hepatic impairment.

FIG. 2 is a semi-logarithmic scale graph of the arithmetic mean plasma concentration-time profiles of rivoceranib following administration of rivoceranib or a pharmaceutically acceptable salt thereof in subjects with normal hepatic function, mild hepatic impairment, and moderate hepatic impairment.

FIGS. 3A and 3B are a linear scale graph and a semi-logarithmic scale graph, respectively, of the overlaid individual pharmacokinetic concentration-time profiles for rivoceranib following administration of rivoceranib in subjects with normal hepatic function.

FIGS. 4A and 4B are a linear scale graph and a semi-logarithmic scale graph, respectively, of the overlaid individual pharmacokinetic concentration-time profiles for rivoceranib following administration of rivoceranib in subjects with mild hepatic impairment.

FIGS. 5A and 5B are a linear scale graph and a semi-logarithmic scale graph, respectively, of the overlaid individual pharmacokinetic concentration-time profiles for rivoceranib following administration of rivoceranib in subjects with moderate hepatic impairment.

### DETAILED DESCRIPTION OF THE DISCLOSURE

Described herein are methods and compositions for treating proliferative diseases, in particular, methods and compositions for treating cancer in subjects with mild or moderate hepatic impairment. The methods include administering a composition including or consisting of rivoceranib or a pharmaceutically acceptable salt thereof to the subject with mild or moderate hepatic impairment. In some embodiments, rivoceranib or a pharmaceutically acceptable salt thereof, is administered in a total daily amount in a range of about 100 mg to about 700 mg. In general, the methods include assessing the hepatic function of a subject or patient or treating a patient or subject who has already been diagnosed with a hepatic impairment. In some embodiments, the methods include determining that the subject has mild or moderate hepatic impairment. In some embodiments, the methods include assessing the hepatic function of the subject; determining that the subject has mild or moderate hepatic impairment; and administering the same therapeutically effective amount of rivoceranib or a pharmaceutically acceptable salt thereof to the subject with mild or moderate hepatic impairment that would be administered to a subject with normal hepatic function.

In general, the methods disclosed herein are used to treat, e.g., improve, cancer in a subject. For example, in some embodiments, the methods can reduce tumor size in a subject or inhibit the growth of a tumor, e.g., as determined by a diameter, area, or volume measurement or estimation based on imaging the tumor, e.g., using known imaging methods such a x-ray, computed tomography (CT), magnetic resonance imaging (MRI), positron emission tomography (PET), and ultrasound.

In some embodiments, the methods and compositions can suppress tumor growth in a subject with cancer, wherein the subject has mild or moderate hepatic impairment. In some embodiments, the methods including administering the same therapeutically effective amount of rivoceranib or a pharmaceutically acceptable salt thereof to the subject with mild or moderate hepatic impairment as would be administered to a subject with normal hepatic function, can suppress tumor growth. In some embodiments, the methods including administering the same therapeutically effective amount of rivoceranib or a pharmaceutically acceptable salt thereof to the subject with mild or moderate hepatic impairment as a subject with normal hepatic function can suppress tumor growth with no treatment-emergent serious adverse events. In some embodiments, the methods including administering the same therapeutically effective amount of rivoceranib or a pharmaceutically acceptable salt thereof to the subject with mild or moderate hepatic impairment as a subject with normal hepatic function can suppress tumor growth with no serious adverse events.

### Rivoceranib (Apatinib)

Generally, rivoceranib is an organic heterocyclic chemical compound of having the following chemical formula:

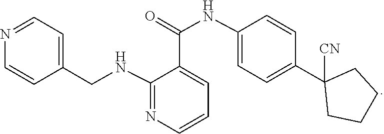 [SMILES: N#CC1(c2ccc(NC(=O)c3cccnc3NCc3ccncc3)cc2)CCCC1]

Rivoceranib (chemical name N-[4-(1-cyanocyclopentyl)phenyl]-2-{[(pyridin-4-yl)methyl]amino} pyridine-3-carboxamide, also known as YN968Dl, developed in China as apatinib and marketed as Aitan®) is an orally administered small molecule tyrosine kinase inhibitor. It selectively inhibits vascular endothelial growth factor receptor (VEGFR)-2 leading to blockage of tumor vascular angiogenesis, diminishes survival of existing blood vessels, and retards growth of tumors. Proliferation of endothelial cells is targeted directly, and inhibition of the release of proangiogenic growth factors by cancer or stromal cells is targeted indirectly.

Recently, an in vitro cytochrome (CYP) phenotyping and metabolism study was performed. In this study, more than 60% of rivoceranib was consumed by CYP3A4 and CYP2D6 at 60 minute incubation, while less than 10% of rivoceranib was consumed by CYP1A2, CYP2B6, CYP2C8, CYP2C9, and CYP2C19. The study results are summarized in Table 1.

TABLE 1
Rivoceranib In Vitro CYP Phenotyping Study Result

| | | | CLINT | | |
|---|---|---|---|---|---|
| | Test article | Recombinant | (μL/minunte/ | t½ | % Remaining at 60 |
| Test article | concentration | CYP isoform | mg) | (minutes) | minute incubation |
| Rivoceranib | 5 μM | CYP1A2 | 0.041 | 671 | 94.7% |
| | | CYP2B6 | 0.020 | 344 | 96.3% |
| | | CYP2C8 | 0.037 | 753 | 103% |
| | | CYP2C9 | 0.119 | 234 | 90.3% |
| | | CYP2C19 | 0.057 | 483 | 98.9% |
| | | CYP2D6 | 0.587 | 47.2 | 34.9% |
| | | CYP3A4 | 0.704 | 39.4 | 37.6% |

Abbreviations: CYP = cytochrome P450; t_{1/2} = apparent terminal elimination half-life.
Source: Cyprotex Study CYP1493-R4 Report, Table 4.2 and Table 5.1

The data in Table 1 indicate that the CYP3A4 and CYP2D6 are major metabolic pathways with possible minor metabolism via CYP2C9. Based on this study, hepatic impairment is expected to significantly impact the PK of rivoceranib and change the unbound plasma concentrations of rivoceranib based on data obtained through previous clinical studies. Although approximately 59.0% of rivoceranib dose is excreted unchanged via feces, the systematically available rivoceranib is extensively metabolized primarily by CY3A4/5 and to a lesser extent by CYP2D6, CYP2C9, and CYP2E1, which are primarily expressed in hepatocytes. In addition, plasma protein binding of rivoceranib is high (97%).

Despite the results of the in vitro cytochrome (CYP) phenotyping and metabolism study described above, it has been unexpectedly and advantageously found that subjects with mild or moderate hepatic impairment administered rivoceranib or a pharmaceutically acceptable salt thereof have similar exposure of rivoceranib as a subject with normal hepatic impairment who is administered the same amount of rivoceranib or a pharmaceutically acceptable salt thereof.

In some embodiments, the exposure is measured by one or more of Cₘₐₓ, AUC₀₋ₜ, and AUC_{0-∞}. In some embodiments, the exposure is measured by Cₘₐₓ, AUC₀₋ₜ, or AUC_{0-∞}. In some embodiments, the subject with mild or moderate hepatic impairment does not have a statistically significant difference in exposure of rivoceranib, than compared to exposure in a subject with normal hepatic function who is administered the same amount of rivoceranib or a pharmaceutically acceptable salt thereof.

As used herein, when the subject does not have a “statistically significant difference in exposure,” that phrase refers to the exposure of a subject is not confirmed as 90% confidence interval for the geometric least square means (GLSM) ratio of rivoceranib Cₘₐₓ and AUCs extending beyond 80%-125% boundary between the subjects with hepatic impairment and subjects with normal hepatic function.

For example, a statistically significant difference in exposure can be measured as follows: a subject with normal hepatic function is matched to 1 subject with mild or moderate hepatic impairment, a paired t-test is applied to analyze the natural log-transformed primary pharmacokinetic (PK) parameters (Cₘₐₓ, AUC₀₋ₜ, and AUC_{0-∞}) for rivoceranib. The paired t-test is performed separately for each hepatic impairment group and the corresponding matched subject with normal hepatic function. Estimates of geometric mean ratios in primary PK parameters between each level of impaired hepatic function versus the matched healthy controls can be analyzed along with the corresponding 90% confidence intervals (CIs) for the geometric least square mean ratios. The p-value assessing the difference between each hepatic impairment group and the normal hepatic function control group is analyzed and the statistically significant difference is determined. In some embodiments, the difference in exposure within subjects after administration of rivoceranib or a pharmaceutically acceptable salt thereof between subjects with normal hepatic function compared with subjects with mild or moderate hepatic impairment is not confirmed.

Provided herein are methods and compositions for treating cancer in a subject or patient with mild or moderate hepatic impairment. The methods include administering a composition including or consisting of rivoceranib or a pharmaceutically acceptable salt thereof to the subject with mild or moderate hepatic impairment. In some embodiments, rivoceranib or a pharmaceutically acceptable salt thereof is administered in a total daily amount in a range of about 100 mg to about 700 mg. In some embodiments, the cancer is improved or inhibited. As used herein, the amount of rivoceranib or a pharmaceutically acceptable salt thereof is provided in the mass of the rivoceranib free base form, even though the rivoceranib can be provided as a pharmaceutically acceptable salt thereof.

For example, when the disclosure describes the amount of rivoceranib or a pharmaceutically acceptable salt thereof administered is about 700 mg, the actual drug administered could be 700 mg of rivoceranib free base or the corresponding amount of rivoceranib pharmaceutically acceptable salt thereof, which would be about 869 mg of a rivoceranib mesylate salt. As another example, when the disclosure describes the amount of rivoceranib or a pharmaceutically acceptable salt thereof administered is about 200 mg, the actual drug administered could be 200 mg of rivoceranib free base or the corresponding amount of rivoceranib pharmaceutically acceptable salt thereof, which would be about 248 mg of a rivoceranib mesylate salt. In another example, when the disclosure describes the amount of rivoceranib or a pharmaceutically acceptable salt thereof administered is about 100 mg, the actual drug administered could be 100 mg of rivoceranib free base or the corresponding amount of rivoceranib pharmaceutically acceptable salt thereof, which would be about 124 mg of a rivoceranib mesylate salt.

In some embodiments, the rivoceranib is administered in the form of a free base. In some embodiments, the rivoceranib is administered in the form of a pharmaceutically acceptable salt, such as a mesylate salt form, e.g., having the following chemical structure:

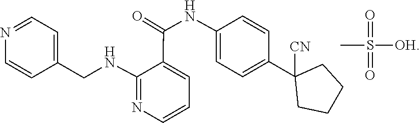

The rivoceranib mesylate has a CAS Registry no. 1218779-75-9, molecular weight of 493.58, and empirical formula C₂₅H₂₇N₅O₄S.

Suitable examples of pharmaceutically acceptable salts include metal salts, such as sodium salts, potassium salts, and lithium salts; alkaline earth metals, such as calcium salts, magnesium salts, and the like; organic amine salts, such as triethylamine salts, pyridine salts, picoline salts, ethanolamine salts, triethanolamine salts, dicyclohexylamine salts, N,N′-dibenzylethylenediamine salts, and the like; inorganic acid salts such as hydrochloride salts, hydrobromide salts, sulfate salts, phosphate salts, and the like; organic acid salts such as formate salts, acetate salts, trifluoroacetate salts, maleate salts, tartrate salts, and the like; sulfonate salts such as methanesulfonate salts, benzenesulfonate salts, p-toluenesulfonate salts, and the like; and amino acid salts, such as arginate salts, asparginate salts, glutamate salts, and the like. Pharmaceutically acceptable salts also include bitartrate, bitartrate hydrate, bitartrate hemipentahydrate, pentafluoropropionate, mucate, oleate, phosphate dibasic, phosphate monobasic, acetate trihydrate, bis(heptafuorobutyrate), bis(pentafluoropropionate), bis(pyridine carboxylate), bis(trifluoroacetate), chlorhydrate, and sulfate pentahydrate. Other representative pharmaceutically acceptable salts include, e.g., water-soluble and water-insoluble salts, such as the amsonate(4,4-diaminostilbene-2,2-disulfonate), benzonate, bicarbonate, bisulfate, borate, butyrate, calcium edetate, camphorsulfonate, camsylate, carbonate, citrate, clavulariate, dihydrochloride, edetate, edisylate, estolate, esylate, fumarate, fumarate, gluceptate, gluconate, glycollylarsanilate, hexafluorophosphate, hexylresorcinate, hydrabamine, hydroxynaphthoate, iodide, isothionate, lactate, lactobionate, laurate, malate, mandelate, mesylate, methylbromide, methylnitrate, methylsulfate, napsylate, nitrate, N-methylglucamine ammonium salt, 3-hydroxy-2-naphthoate, oxalate, palmitate, pamoate, pantothenate, phosphate/diphosphate, picrate, polygalacturonate, propionate, salicylate, stearate, succinate, sulfosalicylate, suramate, tannate, teoclate, tosylate, triethiodide, and valerate salts.

In some embodiments, the methods of treating cancer in a subject with mild or moderate hepatic impairment include assessing hepatic function of the subject; determining that the subject has mild or moderate hepatic impairment; and administering the same therapeutically effective amount of rivoceranib or a pharmaceutically acceptable salt thereof to the subject with mild or moderate hepatic impairment as a subject with normal hepatic function. In some embodiments, the therapeutically effective amount of rivoceranib or a pharmaceutically acceptable salt thereof is administered in a total daily dose or amount in a range of about 100 mg to about 700 mg. In some embodiments, the cancer is improved or at least growth of the cancer is inhibited.

In some embodiments, the rivoceranib or a pharmaceutically acceptable salt thereof is administered, e.g., in a total daily dose, in an amount of at least 100 mg. In some embodiments, the rivoceranib or a pharmaceutically acceptable salt thereof is administered, e.g., in a total daily dose, in an amount of at least 150 mg. In some embodiments, the rivoceranib or a pharmaceutically acceptable salt thereof is administered, e.g., in a total daily dose, in an amount of at least 200 mg. In some embodiments, the rivoceranib or a pharmaceutically acceptable salt thereof is administered, e.g., in a total daily dose, in an amount of at least 225 mg. In some embodiments, the rivoceranib or a pharmaceutically acceptable salt thereof is administered, e.g., in a total daily dose, in an amount of at least 250 mg. In some embodiments, the rivoceranib or a pharmaceutically acceptable salt thereof is administered, e.g., in a total daily dose, in an amount of at least 275 mg.

In some embodiments, the rivoceranib or a pharmaceutically acceptable salt thereof is administered, e.g., in a total daily dose, in an amount of at least 300 mg. In some embodiments, the rivoceranib or a pharmaceutically acceptable salt thereof is administered, e.g., in a total daily dose, in an amount of at least 325 mg. In some embodiments, the rivoceranib or a pharmaceutically acceptable salt thereof is administered, e.g., in a total daily dose, in an amount of at least 350 mg. In some embodiments, the rivoceranib or a pharmaceutically acceptable salt thereof is administered, e.g., in a total daily dose, in an amount of at least 375 mg.

In some embodiments, the rivoceranib or a pharmaceutically acceptable salt thereof is administered, e.g., in a total daily dose, in an amount of at least 400 mg. In some embodiments, the rivoceranib or a pharmaceutically acceptable salt thereof is administered, e.g., in a total daily dose, in an amount of at least 410 mg. In some embodiments, the rivoceranib or a pharmaceutically acceptable salt thereof is administered, e.g., in a total daily dose, in an amount of at least 420 mg. In some embodiments, the rivoceranib or a pharmaceutically acceptable salt thereof is administered, e.g., in a total daily dose, in an amount of at least 425 mg. In some embodiments, the rivoceranib or a pharmaceutically acceptable salt thereof is administered, e.g., in a total daily dose, in an amount of at least 430 mg. In some embodiments, the rivoceranib or a pharmaceutically acceptable salt thereof is administered, e.g., in a total daily dose, in an amount of at least 440 mg. In some embodiments, the rivoceranib or a pharmaceutically acceptable salt thereof is administered, e.g., in a total daily dose, in an amount of at least 450 mg. In some embodiments, the rivoceranib or a pharmaceutically acceptable salt thereof is administered, e.g., in a total daily dose, in an amount of at least 460 mg. In some embodiments, the rivoceranib or a pharmaceutically acceptable salt thereof is administered, e.g., in a total daily dose, in an amount of at least 470 mg. In some embodiments, the rivoceranib or a pharmaceutically acceptable salt thereof is administered, e.g., in a total daily dose, in an amount of at least 475 mg. In some embodiments, the rivoceranib or a pharmaceutically acceptable salt thereof is administered, e.g., in a total daily dose, in an amount of at least 480 mg. In some embodiments, the rivoceranib or a pharmaceutically acceptable salt thereof is administered, e.g., in a total daily dose, in an amount of at least 490 mg.

In some embodiments, the rivoceranib or a pharmaceutically acceptable salt thereof is administered, e.g., in a total daily dose, in an amount of at least 500 mg. In some embodiments, the rivoceranib or a pharmaceutically acceptable salt thereof is administered, e.g., in a total daily dose, in an amount of at least 510 mg. In some embodiments, the rivoceranib or a pharmaceutically acceptable salt thereof is administered, e.g., in a total daily dose, in an amount of at least 520 mg. In some embodiments, the rivoceranib or a pharmaceutically acceptable salt thereof is administered, e.g., in a total daily dose, in an amount of at least 525 mg. In some embodiments, the rivoceranib or a pharmaceutically acceptable salt thereof is administered, e.g., in a total daily dose, in an amount of at least 530 mg. In some embodiments, the rivoceranib or a pharmaceutically acceptable salt thereof is administered, e.g., in a total daily dose, in an amount of at least 540 mg. In some embodiments, the rivoceranib or a pharmaceutically acceptable salt thereof is administered, e.g., in a total daily dose, in an amount of at least 550 mg. In some embodiments, the rivoceranib or a pharmaceutically acceptable salt thereof is administered, e.g., in a total daily dose, in an amount of at least 560 mg. In some embodiments, the rivoceranib or a pharmaceutically acceptable salt thereof is administered, e.g., in a total daily dose, in an amount of at least 570 mg. In some embodiments, the rivoceranib or a pharmaceutically acceptable salt thereof is administered, e.g., in a total daily dose, in an amount of at least 575 mg. In some embodiments, the rivoceranib or a pharmaceutically acceptable salt thereof is administered, e.g., in a total daily dose, in an amount of at least 580 mg. In some embodiments, the rivoceranib or a pharmaceutically acceptable salt thereof is administered, e.g., in a total daily dose, in an amount of at least 590 mg.

In some embodiments, the rivoceranib or a pharmaceutically acceptable salt thereof is administered, e.g., in a total daily dose, in an amount of at least 600 mg. In some embodiments, the rivoceranib or a pharmaceutically acceptable salt thereof is administered, e.g., in a total daily dose, in an amount of at least 610 mg. In some embodiments, the rivoceranib or a pharmaceutically acceptable salt thereof is administered, e.g., in a total daily dose, in an amount of at least 620 mg. In some embodiments, the rivoceranib or a pharmaceutically acceptable salt thereof is administered, e.g., in a total daily dose, in an amount of at least 625 mg. In some embodiments, the rivoceranib or a pharmaceutically acceptable salt thereof is administered, e.g., in a total daily dose, in an amount of at least 630 mg. In some embodiments, the rivoceranib or a pharmaceutically acceptable salt thereof is administered, e.g., in a total daily dose, in an amount of at least 640 mg. In some embodiments, the rivoceranib or a pharmaceutically acceptable salt thereof is administered, e.g., in a total daily dose, in an amount of at least 650 mg. In some embodiments, the rivoceranib or a pharmaceutically acceptable salt thereof is administered, e.g., in a total daily dose, in an amount of at least 660 mg. In some embodiments, the rivoceranib or a pharmaceutically acceptable salt thereof is administered, e.g., in a total daily dose, in an amount of at least 670 mg. In some embodiments, the rivoceranib or a pharmaceutically acceptable salt thereof is administered, e.g., in a total daily dose, in an amount of at least 675 mg. In some embodiments, the rivoceranib or a pharmaceutically acceptable salt thereof is administered, e.g., in a total daily dose, in an amount of at least 680 mg. In some embodiments, the rivoceranib or a pharmaceutically acceptable salt thereof is administered, e.g., in a total daily dose, in an amount of at least 690 mg.

In some embodiments, the rivoceranib or a pharmaceutically acceptable salt thereof is administered, e.g., in a total daily dose, in an amount of at least 700 mg. In some embodiments, the rivoceranib or a pharmaceutically acceptable salt thereof is rivoceranib. In some embodiments, the rivoceranib or a pharmaceutically acceptable salt thereof is a pharmaceutically acceptable salt of rivoceranib. In some embodiments, the rivoceranib or a pharmaceutically acceptable salt thereof is rivoceranib mesylate.

In some embodiments, the rivoceranib or a pharmaceutically acceptable salt thereof is administered in an amount of from about 100 mg to about 700 mg. In some embodiments, the rivoceranib or a pharmaceutically acceptable salt thereof is administered in an amount of from 100 mg to 900 mg. In some embodiments, the rivoceranib or a pharmaceutically acceptable salt thereof is administered in an amount of from 150 mg to 850 mg. In some embodiments, the rivoceranib or a pharmaceutically acceptable salt thereof is administered in an amount of from 175 mg to 825 mg. In some embodiments, the rivoceranib or a pharmaceutically acceptable salt thereof is administered in an amount of from 175 mg to 800 mg. In some embodiments, the rivoceranib or a pharmaceutically acceptable salt thereof is administered in an amount of from 175 mg to 700 mg. In some embodiments, the rivoceranib or a pharmaceutically acceptable salt thereof is administered in an amount of from 175 mg to 775 mg. In some embodiments, the rivoceranib or a pharmaceutically acceptable salt thereof is administered in an amount of from 175 mg to 750 mg. In some embodiments, the rivoceranib or a pharmaceutically acceptable salt thereof is administered in an amount of from 175 mg to 700 mg. In some embodiments, the rivoceranib or a pharmaceutically acceptable salt thereof is administered in an amount of from 175 mg to 600 mg. In some embodiments, the rivoceranib or a pharmaceutically acceptable salt thereof is administered in an amount of from 175 mg to 550 mg. In some embodiments, the rivoceranib or a pharmaceutically acceptable salt thereof is administered in an amount of from 175 mg to 500 mg. In some embodiments, the rivoceranib or a pharmaceutically acceptable salt thereof is administered in an amount of from 175 mg to 400 mg. In some embodiments, the rivoceranib or a pharmaceutically acceptable salt thereof is administered in an amount of from 175 mg to 350 mg. In some embodiments, the rivoceranib or a pharmaceutically acceptable salt thereof is administered in an amount of from 175 mg to 300 mg. In some embodiments, the rivoceranib or a pharmaceutically acceptable salt thereof is administered in an amount of from 200 mg to 700 mg. In some embodiments, the rivoceranib or a pharmaceutically acceptable salt thereof is administered in an amount of from 200 mg to 600 mg. In some embodiments, the rivoceranib or a pharmaceutically acceptable salt thereof is administered in an amount of from 200 mg to 550 mg. In some embodiments, the rivoceranib or a pharmaceutically acceptable salt thereof is administered in an amount of from 200 mg to 500 mg. In some embodiments, the rivoceranib or a pharmaceutically acceptable salt thereof is administered in an amount of from 200 mg to 400 mg.

In some embodiments, rivoceranib or a pharmaceutically acceptable salt thereof is administered in an amount of 500 mg or less. In some embodiments, rivoceranib or a pharmaceutically acceptable salt thereof is administered in an amount of 200 mg or more.

In some embodiments, the rivoceranib or a pharmaceutically acceptable salt thereof is administered orally. In some embodiments, the rivoceranib or a pharmaceutically acceptable salt thereof is administered in an oral liquid, solid or semisolid dosage form. In some embodiments, the rivoceranib or a pharmaceutically acceptable salt thereof is administered as a solid oral dosage form. In some embodiments, the rivoceranib or a pharmaceutically acceptable salt thereof is administered as a pill, tablet, chewable tablet, specialty tablet, buccal tablet, sub-lingual tablet, orally disintegrating tablet, capsule, gel capsule, soft gel capsule, hard gel capsule, sachet, powder, granule, crystal or orally dispersible film. In some embodiments, the rivoceranib or a pharmaceutically acceptable salt thereof is administered as a dried powder, a liquid, a capsule, a pellet or a tablet. In some embodiments, the rivoceranib or a pharmaceutically acceptable salt thereof is administered as a tablet. In some embodiments, the rivoceranib or a pharmaceutically acceptable salt thereof is administered as a film coated tablet.

In such embodiments, wherein the rivoceranib or a pharmaceutically acceptable salt thereof is administered as a solid oral dosage form, the rivoceranib or a pharmaceutically acceptable salt thereof may be admixed with at least one inert customary excipient (or carrier) such as sodium citrate or dicalcium phosphate or (a) fillers or extenders, for example, starches, lactose, sucrose, glucose, mannitol, and silicic acid, (b) binders, for example, cellulose derivatives, starch, alignates, gelatin, polyvinylpyrrolidone, sucrose, and gum acacia, (c) humectants, for example, glycerol, (d) disintegrating agents, for example, agar, calcium carbonate, potato or tapioca starch, alginic acid, croscarmellose sodium, complex silicates, and sodium carbonate, (e) solution retarders, for example, paraffin, (f) absorption accelerators, for example, quaternary ammonium compounds, (g) wetting agents, for example, cetyl alcohol, glycerol monostearate, magnesium stearate and the like, (h) adsorbents, for example, kaolin and bentonite, and (i) lubricants, for example, talc, calcium stearate, magnesium stearate, solid polyethylene glycols, sodium lauryl sulfate, or mixtures thereof. In the case of capsules, tablets, and pills, the dosage forms may also comprise buffering agents.

In some embodiments, solid dosage forms may be prepared with coatings and shells, such as enteric coatings and others known in the art. They may contain pacifying agents, and can also be of such composition that they release the rivoceranib or a pharmaceutically acceptable salt thereof in a certain part of the intestinal tract in a delayed manner. Examples of embedded compositions that can be used are polymeric substances and waxes. The rivoceranib or a pharmaceutically acceptable salt thereof may also be in microencapsulated form, if appropriate, with one or more of the above-mentioned excipients.

In some embodiments, the tablet comprises the rivoceranib or a pharmaceutically acceptable salt thereof in an amount of about 100 mg. In some embodiments, the tablet comprises the rivoceranib or a pharmaceutically acceptable salt thereof in an amount of about 150 mg. In some embodiments, the tablet comprises the rivoceranib or a pharmaceutically acceptable salt thereof in an amount of about 200 mg. In some embodiments, the tablet comprises the rivoceranib or a pharmaceutically acceptable salt thereof in an amount of about 250 mg. In some embodiments, the tablet comprises the rivoceranib or a pharmaceutically acceptable salt thereof in an amount of about 300 mg. In some embodiments, the tablet comprises the rivoceranib or a pharmaceutically acceptable salt thereof in an amount of about 350 mg.

In some embodiments, the tablet comprises the rivoceranib or a pharmaceutically acceptable salt thereof in an amount of about 400 mg. In some embodiments, the tablet comprises the rivoceranib or a pharmaceutically acceptable salt thereof in an amount of about 450 mg. In some embodiments, the tablet comprises the rivoceranib or a pharmaceutically acceptable salt thereof in an amount of about 500 mg. In some embodiments, the tablet comprises the rivoceranib or a pharmaceutically acceptable salt thereof in an amount of about 550 mg. In some embodiments, the tablet comprises the rivoceranib or a pharmaceutically acceptable salt thereof in an amount of about 600 mg. In some embodiments, the tablet comprises the rivoceranib or a pharmaceutically acceptable salt thereof in an amount of about 650 mg. In some embodiments, the tablet further comprises one or more of pregelatinized starch, microcrystalline cellulose, sodium starch glycolate, povidone (K-30), colloidal silicon dioxide, magnesium stearate and Opadry white.

In some embodiments, the rivoceranib or a pharmaceutically acceptable salt thereof is administered in one or more 100 mg rivoceranib tablets. In some embodiments, the rivoceranib or a pharmaceutically acceptable salt thereof is administered in two, three, four, five, six, or seven 100 mg rivoceranib tablets. In some embodiments, the rivoceranib or a pharmaceutically acceptable salt thereof is administered in one or more 200 mg rivoceranib tablets. In some embodiments, the rivoceranib or a pharmaceutically acceptable salt thereof is administered in two, three, or four 200 mg rivoceranib tablets. In some embodiments, the rivoceranib or a pharmaceutically acceptable salt thereof is administered in 200 mg rivoceranib tablets and 100 mg rivoceranib tablets. In some embodiments, the rivoceranib or a pharmaceutically acceptable salt thereof is administered in one 200 mg rivoceranib tablet and one 100 mg rivoceranib tablets. In some embodiments, the rivoceranib or a pharmaceutically acceptable salt thereof is administered in one 200 mg rivoceranib tablet and two 100 mg rivoceranib tablets. In some embodiments, the rivoceranib or a pharmaceutically acceptable salt thereof is administered in one 200 mg rivoceranib tablet and three 100 mg rivoceranib tablets. In some embodiments, the rivoceranib or a pharmaceutically acceptable salt thereof is administered in two 200 mg rivoceranib tablets and one 100 mg rivoceranib tablet. In some embodiments, the rivoceranib or a pharmaceutically acceptable salt thereof is administered in two 200 mg rivoceranib tablets and two 100 mg rivoceranib tablets. In some embodiments, the rivoceranib or a pharmaceutically acceptable salt thereof is administered in two 200 mg rivoceranib tablets and three 100 mg rivoceranib tablets. In some embodiments, the rivoceranib or a pharmaceutically acceptable salt thereof is administered in three 200 mg rivoceranib tablets and one 100 mg rivoceranib tablets.

In some embodiments, the rivoceranib or a pharmaceutically acceptable salt thereof is administered as a liquid oral dosage form. In some embodiments, the rivoceranib or a pharmaceutically acceptable salt thereof is administered as a solution, suspension, drink, syrup, elixir, ampoule, dispersion, semi-solid or soft gel.

In some embodiments, the rivoceranib or a pharmaceutically acceptable salt thereof is administered parenterally. In some embodiments, the rivoceranib or a pharmaceutically acceptable salt thereof is administered intradermaly, subcutaneously, intramuscularly, intraosseously, intraperitoneally or intravenously. In some embodiments, the rivoceranib or a pharmaceutically acceptable salt thereof is administered intraperitoneally. In some embodiments, the rivoceranib or a pharmaceutically acceptable salt thereof is administered intravenously.

### Dosing

In some instances, drug dosages are determined as a factor of patient body surface area (BSA). In some instances, BSA is a better indicator of metabolic mass than body weight because it is less affected by abnormal adipose mass, e.g., a patient with a larger BSA would presumably have larger organs for a drug to clear through. Indeed, there can be a 4 to 10-fold variation in drug clearance between individuals.

Various formulae exist, using height and weight, to calculate BSA without direct measurement. The most widely used is the Du Bois formula, which has been shown to be equally as effective in estimating BSA in obese and non-obese patients.

$$BSA = 0.007184 \times {W}^{0.425} \times {H}^{0.725}$$

where W is mass in kg, and H is height in cm. The average adult male BSA is 2.060 m². The average adult female BSA is 1.830 m².

In some instances, drug dosages can be determined based upon the subject's response to the drug, e.g., adverse events other than disease progression. For example, when a subject is administered a dose of the drug or multiple doses of the drug and exhibits an adverse event other than disease progression, the dosage of the drug may be reduced or the dosage of the drug may be interrupted, e.g., the dosage of the drug is no longer administered daily and can be postponed days without administration or can be administered every other day or less.

In some embodiments, the rivoceranib or a pharmaceutically acceptable salt thereof is administered once daily. In some embodiments, the rivoceranib or a pharmaceutically acceptable salt thereof is administered twice daily.

In some embodiments, the rivoceranib or a pharmaceutically acceptable salt thereof is administered once daily in a total daily amount of about 200 mg. In some embodiments, the rivoceranib or a pharmaceutically acceptable salt thereof is administered twice daily in a total daily amount of about 200 mg. In some embodiments, the rivoceranib or a pharmaceutically acceptable salt thereof is administered once daily in a total daily amount of about 500 mg, 300 mg, or 200 mg. In some embodiments, the rivoceranib or a pharmaceutically acceptable salt thereof is administered once daily in a total daily amount of about 300 mg. In some embodiments, the rivoceranib or a pharmaceutically acceptable salt thereof is administered once daily in a total daily amount of about 500 mg.

Described herein are dosing regimens for rivoceranib or a pharmaceutically acceptable salt thereof in treatment of cancer in a subject with a mild or moderate hepatic impairment comprising rivoceranib or a pharmaceutically acceptable salt thereof. In some embodiments, a total daily amount is administered in a range of about 100 mg to about 700 mg for at least one cycle, wherein each cycle is at least 10 days. In some embodiments, the dosing regimen for rivoceranib or a pharmaceutically acceptable salt thereof in treatment of cancer in a subject with a mild or moderate hepatic impairment comprising rivoceranib or a pharmaceutically acceptable salt thereof, wherein a total daily amount is administered in about 200 mg for at least one cycle. In some embodiments, the dosing regimen for rivoceranib or a pharmaceutically acceptable salt thereof in treatment of cancer in a subject with a mild or moderate hepatic impairment comprising rivoceranib or a pharmaceutically acceptable salt thereof, wherein a total daily amount is administered in about 300 mg for at least one cycle. In some embodiments, the dosing regimen for rivoceranib or a pharmaceutically acceptable salt thereof in treatment of cancer in a subject with a mild or moderate hepatic impairment comprising rivoceranib or a pharmaceutically acceptable salt thereof, wherein a total daily amount is administered in about 500 mg for at least one cycle. In some embodiments, the cycle is at least 10, 14, 15, 20, 21, 28, 29, 30, or 31 days. In some embodiments, the cycle is at least 28 days. In some embodiments, the cycle is 28 days. In some embodiments, the rivoceranib or a pharmaceutically acceptable salt thereof is administered in a total daily amount of about 200 mg for at least two cycles, at least three cycles, at least four cycles, at least five cycles, or at least six cycles.

### Cancers

Described herein are methods of treating cancer in a subject with mild or moderate hepatic impairment, where the methods include administering rivoceranib or a pharmaceutically acceptable salt thereof. In some embodiment, the cancer is improved.

Described herein are methods of treating cancer in a subject with mild or moderate hepatic impairment, wherein the methods include assessing hepatic function of the subject; determining that the subject has mild or moderate hepatic impairment; and administering the same therapeutically effective amount of rivoceranib or a pharmaceutically acceptable salt thereof to the subject with mild or moderate hepatic impairment as a subject with normal hepatic function. In some embodiments, the cancer is improved.

In some embodiments, the cancer is selected from Acanthoma, Acinic cell carcinoma, Acoustic neuroma, Acral lentiginous melanoma, Acrospiroma, Acute eosinophilic leukemia, Acute lymphoblastic leukemia, Acute megakaryoblastic leukemia, Acute monocytic leukemia, Acute myeloblastic leukemia with maturation, Acute myeloid dendritic cell leukemia, Acute myeloid leukemia, Acute promyelocytic leukemia, Adamantinoma, Adenocarcinoma, Adenoid cystic carcinoma, Adenoma, Adenomatoid odontogenic tumor, Adrenocortical carcinoma, Adult T-cell leukemia, Aggressive NK-cell leukemia, AIDS-Related Cancers, AIDS-related lymphoma, Alveolar soft part sarcoma, Ameloblastic fibroma, Anal cancer, Anaplastic large cell lymphoma, Anaplastic thyroid cancer, Angioimmunoblastic T-cell lymphoma, Angiomyolipoma, Angiosarcoma, Appendix cancer, Astrocytoma, Atypical teratoid rhabdoid tumor, Basal cell carcinoma, Basal-like carcinoma, B-cell leukemia, B-cell lymphoma, Bellini duct carcinoma, Biliary tract cancer, Bladder cancer, Blastoma, Bone Cancer, Bone tumor, Brain Stem Glioma, Brain Tumor, Breast Cancer, Brenner tumor, Bronchial Tumor, Bronchioloalveolar carcinoma, Brown tumor, Burkitt's lymphoma, Cancer of Unknown Primary Site, Carcinoid Tumor, Carcinoma, Carcinoma in situ, Carcinoma of the penis, Carcinoma of Unknown Primary Site, Carcinosarcoma, Castleman's Disease, Central Nervous System Embryonal Tumor, Cerebellar Astrocytoma, Cerebral Astrocytoma, Cervical Cancer, Cholangiocarcinoma, Chondroma, Chondrosarcoma, Chordoma, Choriocarcinoma, Choroid plexus papilloma, Chronic Lymphocytic Leukemia, Chronic monocytic leukemia, Chronic myelogenous leukemia, Chronic Myeloproliferative Disorder, Chronic neutrophilic leukemia, Clear-cell tumor, Colon Cancer, Colorectal cancer, Craniopharyngioma, Cutaneous T-cell lymphoma, Degos disease, Dermatofibrosarcoma protuberans, Dermoid cyst, Desmoplastic small round cell tumor, Diffuse large B cell lymphoma, Dysembryoplastic neuroepithelial tumor, Embryonal carcinoma, Endodermal sinus tumor, Endometrial cancer, Endometrial Uterine Cancer, Endometrioid tumor, Enteropathy-associated T-cell lymphoma, Ependymoblastoma, Ependymoma, Epithelioid sarcoma, Erythroleukemia, Esophageal cancer, Esthesioneuroblastoma, Ewing's sarcoma, Extracranial Germ Cell Tumor, Extragonadal Germ Cell Tumor, Extrahepatic Bile Duct Cancer, Extramammary Paget's disease, Fallopian tube cancer, Fetus in fetu, Fibroma, Fibrosarcoma, Follicular lymphoma, Follicular thyroid cancer, Gallbladder Cancer, Ganglioglioma, Ganglioneuroma, Gastric Cancer, Gastric lymphoma, Gastrointestinal cancer, Gastrointestinal Carcinoid Tumor, Gastrointestinal Stromal Tumor, Germ cell tumor, Germinoma, Gestational choriocarcinoma, Gestational Trophoblastic Tumor, Giant cell tumor of bone, Glioblastoma multiforme, Glioma, Gliomatosis cerebri, Glomus tumor, Glucagonoma, Gonadoblastoma, Granulosa cell tumor, Hairy cell leukemia, Head and Neck Cancer, Heart cancer, Hemangioblastoma, Hemangiopericytoma, Hemangiosarcoma, Hematological malignancy, Hepatocellular carcinoma, Hepatosplenic T-cell lymphoma, Hereditary breast-ovarian cancer syndrome, Hodgkin's lymphoma, Hypopharyngeal Cancer, Hypothalamic Glioma, Inflammatory breast cancer, Intraocular Melanoma, Islet cell carcinoma, Islet Cell Tumor, Juvenile myelomonocytic leukemia, Kaposi's sarcoma, Kidney Cancer, Klatskin tumor, Krukenberg tumor, Laryngeal Cancer, Laryngeal cancer, Lentigo maligna melanoma, Leukemia, Lip and Oral Cavity Cancer, Liposarcoma, Lung cancer, Luteoma, Lymphangioma, Lymphangiosarcoma, Lymphoepithelioma, Lymphoid leukemia, Lymphoma, Macroglobulinemia, Malignant fibrous histiocytoma, Malignant Fibrous Histiocytoma of Bone, Malignant Glioma, Malignant Mesothelioma, Malignant peripheral nerve sheath tumor, Malignant rhabdoid tumor, Malignant triton tumor, MALT lymphoma, Mantle cell lymphoma, Mast cell leukemia, Mediastinal germ cell tumor, Mediastinal tumor, Medullary thyroid cancer, Medulloblastoma, Medulloepithelioma, Melanoma, Meningioma, Merkel Cell Carcinoma, Mesothelioma, Metastatic Squamous Neck Cancer with Occult Primary, Metastatic urothelial carcinoma, Mixed Mullerian tumor, Monocytic leukemia, Mouth Cancer, Mucinous tumor, Multiple Endocrine Neoplasia Syndrome, Multiple Myeloma, Mycosis fungoides, Myelodysplastic Syndromes, Myeloid leukemia, Myeloid sarcoma, Myeloproliferative Disease, Myxoma, Nasal Cavity Cancer, Nasopharyngeal Cancer, Neoplasm, Neurinoma, Neuroblastoma, Neurofibroma, Neuroma, Nodular melanoma, Non-Hodgkin Lymphoma, Non-melanoma Skin Cancer, Non-Small Cell Lung Cancer, Ocular oncology, Oligoastrocytoma, Oligodendroglioma, Oncocytoma, Optic nerve sheath meningioma, Oral Cancer, Oropharyngeal Cancer, Osteosarcoma, Ovarian Cancer, Ovarian Epithelial Cancer, Ovarian Germ Cell Tumor, Ovarian Low Malignant Potential Tumor, Paget's disease of the breast, Pancoast tumor, Pancreatic cancer, Papillary thyroid cancer, Papillomatosis, Paraganglioma, Paranasal Sinus Cancer, Parathyroid Cancer, Penile Cancer, Perivascular epithelioid cell tumor, Pharyngeal Cancer, Pheochromocytoma, Pineal Parenchymal Tumor of Intermediate Differentiation, Pineoblastoma, Pituicytoma, Pituitary adenoma, Pituitary tumor, Plasma Cell Neoplasm, Pleuropulmonary blastoma, Polyembryoma, Precursor T-lymphoblastic lymphoma, Primary central nervous system lymphoma, Primary effusion lymphoma, Primary Hepatocellular Cancer, Primary Liver Cancer, Primary peritoneal cancer, Primitive neuroectodermal tumor, Prostate cancer, Pseudomyxoma peritonei, Rectal Cancer, Renal cell carcinoma, Respiratory Tract Carcinoma Involving the NUT Gene on Chromosome 15, Retinoblastoma, Rhabdomyoma, Rhabdomyosarcoma, Richter's transformation, Sacrococcygeal teratoma, Salivary Gland Cancer, Sarcoma, Schwannomatosis, Sebaceous gland carcinoma, Secondary neoplasm, Seminoma, Serous tumor, Sertoli-Leydig cell tumor, Sex cord-stromal tumor, Sezary Syndrome, Signet ring cell carcinoma, Skin Cancer, Small blue round cell tumor, Small cell carcinoma, Small Cell Lung Cancer, Small cell lymphoma, Small intestine cancer, Soft tissue sarcoma, Somatostatinoma, Soot wart, Spinal Cord Tumor, Spinal tumor, Splenic marginal zone lymphoma, Squamous cell carcinoma, Stomach cancer, Superficial spreading melanoma, Supratentorial Primitive Neuroectodermal Tumor, Surface epithelial-stromal tumor, Synovial sarcoma, T-cell acute lymphoblastic leukemia, T-cell large granular lymphocyte leukemia, T cell leukemia, T-cell lymphoma, T-cell prolymphocytic leukemia, Teratoma, Terminal lymphatic cancer, Testicular cancer, Thecoma, Throat Cancer, Thymic Carcinoma, Thymoma, Thyroid cancer, Transitional Cell Cancer of Renal Pelvis and Ureter, Transitional cell carcinoma, Urachal cancer, Urethral cancer, Urogenital neoplasm, Uterine sarcoma, Uveal melanoma, Vaginal Cancer, Verner Morrison syndrome, Verrucous carcinoma, Visual Pathway Glioma, Vulvar Cancer, Waldenstrom's macroglobulinemia, Warthin's tumor, Wilms' tumor, or any combination thereof.

In some embodiments, the cancer is liver cancer. In some embodiments, the liver cancer is selected from hepatocellular carcinoma, hepatoma, cholangiocarcinoma, hepatoblastoma, hepatic carcinoma, hepatic angiosarcoma, and metastatic liver cancer. In some embodiments, the cancer is hepatocellular carcinoma.

In some embodiments, the subjects have mild or moderate hepatic impairment that is caused by a disorder or disease other than cancer. In some embodiments, the subjects have mild or moderate hepatic impairment caused by one or more of the disorders or diseases of the group of: liver cirrhosis, chronic liver disease, chronic hepatitis C, chronic hepatitis B, primary sclerosing cholangitis (PSC), splenomegaly, fibrosis stage, varices, liver transplant rejection, delayed function of liver transplant, recurrent disease in transplanted graft, liver injury, and prolonged infection, or the like.

In some embodiments, the cancer comprises one or more lesions. In some embodiments, the lesion is measured before the treatment and either during the treatment or after the treatment or both. In some embodiments, the lesion is measured by radiological assessments using computerized tomography scan or magnetic resonance imaging. In some embodiments, the lesion has reduced in size after the treatment. In some embodiments, the methods include assessing the cancer, wherein one or more lesions are measured before treatment. In some embodiments, the cancer is hepatocellular carcinoma. In some embodiments, the methods include assessing the hepatocellular carcinoma in a subject, wherein assessing includes measuring one or more lesions in the subject, optionally measuring one or more lesions in the subject over a period of time, e.g., 1 week, 1 month, 2 month, 3 months, 4 months, 5 months, 6 months or more.

In some embodiments, the method of treating cancer includes reducing the size of measurable lesions of a subject. In some embodiments, the lesion has reduced in size by at least 10%. In some embodiments, the lesion has reduced in size by at least 20%. In some embodiments, the lesion has reduced in size by at least 25%. In some embodiments, the lesion has reduced in size by at least 30%. In some embodiments, the lesion has reduced in size by at least 40%. In some embodiments, the lesion has reduced in size by at least 50%. In some embodiments, the lesion has reduced in size by at least 60%. In some embodiments, the lesion has reduced in size by at least 70%. In some embodiments, the lesion has reduced in size by at least 75%. In some embodiments, the lesion has reduced in size by at least 80%. In some embodiments, the lesion has reduced in size by at least 90%. In some embodiments, the cancer has improved. In some embodiments, the hepatocellular carcinoma in a subject has improved. In some embodiments, the methods disclosed herein include discontinuing the administration of the rivoceranib or a pharmaceutically acceptable salt thereof when the subject no longer has cancer.

In some embodiments, the methods disclosed herein are a first line of therapy for treating cancer, e.g., hepatocellular carcinoma. In some embodiments, the methods are a second or a third line of therapy after the prior treatment for the cancer has failed or substantially failed or the disease is substantially refractory to the first line therapy. In some embodiments, a patient has received at least one line of therapy for treating cancer prior to being administered rivoceranib or a pharmaceutically acceptable salt thereof. In some embodiments, the prior line of therapy may be a line of chemotherapy or immunotherapy.

In some embodiments, the methods of treating cancer in a subject with mild or moderate hepatic impairment include assessing hepatic function of a subject. In some embodiments, assessing hepatic function of a subject includes classifying the hepatic impairment using the Child-Pugh scale. In some embodiments, the methods include determining that the subject has mild or moderate hepatic impairment, e.g., the subject has a Child-Pugh score of 5-6 or 7-9, respectively. In some embodiments, the methods include administering the same therapeutically effective amount of rivoceranib or a pharmaceutically acceptable salt thereof to the subject with mild or moderate hepatic impairment as to a subject with normal hepatic function. In some embodiments, the subjects having cancer with mild or moderate hepatic impairment are compared to a subject with normal hepatic impairment having similar characteristics (e.g., BSA as described above, body-mass index, demographic, sex, cancer type, etc.) and are administered the same therapeutically effective amount of rivoceranib or a pharmaceutically acceptable salt thereof as the comparable subject with normal hepatic function. For example, a subject with mild or moderate hepatic impairment can be compared to a subject with normal hepatic function if both subjects are demographically matched by age (±10 years), BMI (±20%), and sex. In some embodiments, the subject has mild hepatic impairment. In some embodiments, the subject has moderate hepatic impairment. In some embodiments, the methods include if the subject is determined to have severe hepatic impairment, rivoceranib or a pharmaceutically acceptable salt thereof is not administered to that subject.

In some embodiments, the methods described herein further comprise administering one or more additional agents selected from the group consisting of anti-cancer agents, anti-proliferative agents, chemotherapeutic agents, immunomodulatory agents, anti-angiogenic agents, anti-inflammatory agents, alkylating agents, steroidal and non-steroidal anti-inflammatory agents, pain relievers, leukotriene antagonists, β2-agonists, anticholinergic agents, hormonal agents, biological agents, immunotherapeutic agents, glucocorticoids, corticosteroid agents, antibacterial agents, antihistamines, anti-malarial agents, anti-viral agents, and antibiotics; and, optionally with radiation therapy.

In some embodiments, the rivoceranib or a pharmaceutically acceptable salt thereof is administered for 28 days or more. In some embodiments, the rivoceranib or a pharmaceutically acceptable salt thereof is administered daily for at least 1 or 2 months. In some embodiments, the rivoceranib or a pharmaceutically acceptable salt thereof is administered for about 2 months. In some embodiments, the rivoceranib or a pharmaceutically acceptable salt thereof is administered for about 3 months. In some embodiments, the rivoceranib or a pharmaceutically acceptable salt thereof is administered for about 4 months. In some embodiments, the rivoceranib or a pharmaceutically acceptable salt thereof is administered for about 5 months. In some embodiments, the rivoceranib or a pharmaceutically acceptable salt thereof is administered for about 6 months. In some embodiments, the rivoceranib or a pharmaceutically acceptable salt thereof is administered for about 7 months. In some embodiments, the rivoceranib or a pharmaceutically acceptable salt thereof is administered for about 8 months. In some embodiments, the rivoceranib or a pharmaceutically acceptable salt thereof is administered for about 9 months. In some embodiments, the rivoceranib or a pharmaceutically acceptable salt thereof is administered for about 10 months. In some embodiments, the rivoceranib or a pharmaceutically acceptable salt thereof is administered for about 11 months. In some embodiments, the rivoceranib or a pharmaceutically acceptable salt thereof is administered for about 12 months. In some embodiments, the rivoceranib or a pharmaceutically acceptable salt thereof is administered for 2 months or more.

It is understood that cancer refers to or describes the physiological condition in mammals that is typically characterized by unregulated cell growth. The cancer may be multi-drug resistant (MDR) or drug sensitive.

### Pharmaceutical Compositions

In one aspect, the disclosure relates to pharmaceutical compositions comprising the compounds of the disclosure. That is, a pharmaceutical composition can be provided comprising a therapeutically effective amount of rivoceranib or a pharmaceutically acceptable salt thereof.

In certain aspects, the disclosed pharmaceutical compositions comprise the disclosed compounds (such as rivoceranib or a pharmaceutically acceptable salt thereof) as active ingredients, a pharmaceutically acceptable carrier, and, optionally, other therapeutic ingredients or adjuvants. The instant compositions include those suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

In practice, the compounds of the disclosure, or pharmaceutically acceptable salts thereof, of this disclosure can be combined as the active ingredient in intimate admixture with a pharmaceutical carrier according to conventional pharmaceutical compounding techniques.

The carrier can take a wide variety of forms depending on the form of preparation desired for administration, e.g., oral or parenteral (including intravenous). Thus, the pharmaceutical compositions of the present disclosure can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion or as a water-in-oil liquid emulsion. In addition to the common dosage forms set out above, the compounds of the disclosure, and/or pharmaceutically acceptable salt(s) thereof, can also be administered by controlled release means and/or delivery devices. The compositions can be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

Thus, the pharmaceutical compositions of this disclosure can include a pharmaceutically acceptable carrier and rivoceranib, or a pharmaceutically acceptable salt thereof. The compounds of the disclosure, or pharmaceutically acceptable salts thereof, can also be included in pharmaceutical compositions in combination with one or more other therapeutically active compounds.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In preparing the compositions for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques.

A tablet containing the composition of this disclosure can be prepared by compression or molding, optionally with one or more accessory ingredients or adjuvants. Compressed tablets can be prepared by compressing, in a suitable machine, the active ingredient in a free-flowing form such as powder or granules, optionally mixed with a binder, lubricant, inert diluent, surface active or dispersing agent. Molded tablets can be made by molding in a suitable machine, a mixture of the powdered compound moistened with an inert liquid diluent.

The pharmaceutical compositions of the present disclosure comprise a compound of the disclosure (or pharmaceutically acceptable salts thereof) as an active ingredient, a pharmaceutically acceptable carrier, and optionally one or more additional therapeutic agents or adjuvants. The instant compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

Pharmaceutical compositions of the present disclosure suitable for parenteral administration can be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present disclosure suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Pharmaceutical compositions of the present disclosure can be in a form suitable for topical use such as, for example, an aerosol, cream, ointment, lotion, dusting powder, mouth washes, gargles, and the like. Further, the compositions can be in a form suitable for use in transdermal devices. These formulations can be prepared, utilizing a compound of the disclosure, or pharmaceutically acceptable salts thereof, via conventional processing methods. As an example, a cream or ointment is prepared by mixing hydrophilic material and water, together with about 5 wt % to about 10 wt % of the compound, to produce a cream or ointment having a desired consistency.

Pharmaceutical compositions of this disclosure can be in a form suitable for rectal administration wherein the carrier is a solid. It is preferable that the mixture forms unit dose suppositories. Suitable carriers include cocoa butter and other materials commonly used in the art. The suppositories can be conveniently formed by first admixing the composition with the softened or melted carrier(s) followed by chilling and shaping in moulds.

In addition, the pharmaceutical formulations described above can include, as appropriate, one or more additional carrier ingredients such as diluents, buffers, flavoring agents, binders, surface-active agents, thickeners, lubricants, preservatives (including antioxidants) and the like. Furthermore, other adjuvants can be included to render the formulation isotonic with the blood of the intended recipient. Compositions containing a compound of the disclosure, and/or pharmaceutically acceptable salts thereof, can also be prepared in powder or liquid concentrate form.

The disclosed pharmaceutical compositions can further comprise other therapeutically active compounds, which are usually applied in the treatment of the pathological conditions mentioned above.

In another embodiment, the rivoceranib or a pharmaceutically acceptable salt thereof of the present disclosure comprises more than two compositions contained in the same or separate containers, and these at least two compositions may be administered separately, either simultaneously or sequentially. In another embodiment, the rivoceranib or a pharmaceutically acceptable salt thereof of the present disclosure comprises more than two compositions contained in a blister pack, and these at least two compositions may be administered separately, either simultaneously or sequentially.

### Kits

In some embodiments, kits are provided, comprising: rivoceranib or a pharmaceutically acceptable salt thereof; wherein the kits are for treating cancer in subjects with mild or moderate hepatic impairment.

In some embodiments, kits are provided, comprising about 100 mg to about 700 mg of rivoceranib or a pharmaceutically acceptable salt thereof; wherein the kits are for treating cancer in subjects with mild or moderate hepatic impairment.

In some embodiments, kits are provided, comprising one or more 200 mg tablets comprising rivoceranib or a pharmaceutically acceptable salt thereof; wherein the kits are for treating cancer in subjects with mild or moderate hepatic impairment.

In some embodiments, kits are provided, comprising one 200 mg tablet comprising rivoceranib or a pharmaceutically acceptable salt thereof; wherein the kits are for treating cancer in subjects with mild or moderate hepatic impairment.

In some embodiments, kits are provided, comprising one or more 100 mg tablets comprising rivoceranib or a pharmaceutically acceptable salt thereof; and one or more 200 mg tablets comprising rivoceranib or a pharmaceutically acceptable salt thereof wherein the kits are for treating cancer in subjects with mild or moderate hepatic impairment.

### Methods of Treatment

Also described herein are methods of treating cancer (e.g., hepatocellular carcinoma) in a subject with mild or moderate hepatic impairment, the method comprising: administering the same therapeutically effective amount of rivoceranib or a pharmaceutically acceptable salt thereof to the subject with mild or moderate hepatic impairment as a subject with normal hepatic function, wherein the therapeutically effective amount of rivoceranib or a pharmaceutically acceptable salt thereof is administered orally, in tablet form, in a total daily amount of about 100 mg to about 700 mg, once daily, and wherein rivoceranib or a pharmaceutically acceptable salt thereof is administered as rivoceranib mesylate salt. In some embodiments, the total daily amount of rivoceranib a pharmaceutically acceptable salt thereof administered is about 100 mg, about 200 mg, about 300 mg, or about 500 mg. In some embodiments, the total daily amount of rivoceranib a pharmaceutically acceptable salt thereof administered is about 200 mg.

The methods and compositions disclosed herein are further described in the following examples, which do not limit the scope of the claims.

### EXAMPLES

The following example includes clinical trials for rivoceranib comparing subjects that were administered rivoceranib, wherein the subjects with normal hepatic function are compared to subjects with mild or moderate hepatic impairments to understand the safety, tolerability, and pharmacokinetics of subjects having mild or moderate hepatic impairments.

### Example 1—Phase I Clinical Trials of Rivoceranib

Overall design: A Phase 1, open-label, nonrandomized, single-dose study to investigate the safety, tolerability, and pharmacokinetics of rivoceranib administered 200 mg in subjects with varying degrees of hepatic impairment compared to subjects with normal hepatic function.

Objectives: Primary objectives are to evaluate the plasma pharmacokinetic (PK) profile of a single dose of rivoceranib in subjects with impaired hepatic function compared to control healthy subjects. Secondary objectives are to evaluate the plasma PK profile of rivoceranib metabolites M1-1, M1-2, M1-6, and M9-2 in subjects with impaired hepatic function compared to control healthy subjects after a single dose of rivoceranib, and to evaluate the safety and tolerability of a single dose of rivoceranib in subjects with impaired hepatic function compared to control healthy subjects.

Patients: 27 subjects were enrolled within the following groups based on their Child-Pugh (CP) classification score:
Group 1: Matched-control healthy subjects with normal hepatic function;
Group 2: Subjects with mild hepatic impairment (CP Class A, score of 5 or 6);
Group 3: Subjects with moderate hepatic impairment (CP Class B, score of 7 to 9).

A parallel design strategy was adopted for the hepatic impairment groups. Each matched-control healthy subject (Group 1) was enrolled following the enrollment of a mild and/or moderate hepatic impairment subject and was demographically matched by age (±10 years), BMI (±20%), and sex to the enrolled hepatic impairment subject(s). Subjects with normal hepatic function were not matched to more than 1 hepatically-impaired subject within an impairment group; however, subjects with normal hepatic function could be matched to 1 subject from more than 1 hepatic impairment group.

Subjects were screened to assess their eligibility to enter the study within 35 days prior to dose administration. Subjects were admitted into the Clinical Research Unit (CRU) on Day −1 and confined to the CRU until Discharge on Day 3. Subjects received a follow-up telephone call on Day 7 of the study (±2 days).

Serial blood collections were obtained from predose through 48 hours postdose for analysis of plasma concentrations of rivoceranib and metabolites. Blood samples for rivoceranib plasma protein binding were collected.

Safety was monitored through recording of adverse events (AEs), clinical laboratory evaluations (clinical chemistry, hematology, coagulation parameters, and urinalysis), vital sign measurements, 12-lead electrocardiograms (ECGs), and physical examination findings during the study.

The mean age was 58.1 years in the normal hepatic function group, 63.3 years in the Mild hepatic impairment group and 58.6 years in the Moderate hepatic impairment group. The majority of subjects were male (70.4% overall). The majority of the subjects were not Hispanic or Latino ethnicity (51.9%). Most subjects were white (77.8%). The mean BMI was 29.46 kg/m² in the normal hepatic function group, 28.78 kg/m² in the mild hepatic impairment group and 31.44 kg/m² in the moderate hepatic impairment group.

TABLE 2
Summary of Subject Disposition and Population Assignment

| | Normal | | Moderate | |
|---|---|---|---|---|
| | Hepatic | Mild Hepatic | Hepatic | |
| Population [n (%)] | Function | Impairment | Impairment | Overall |
| All Subjects | 11 | 8 | 8 | 27 |
| | (100.0%) | (100.0%) | (100.0%) | (100.0%) |
| Safety | 11 | 8 | 8 | 27 |
| | (100.0%) | (100.0%) | (100.0%) | (100.0%) |
| Pharmacokinetic | 11 | 8 | 8 | 27 |
| | (100.0%) | (100.0%) | (100.0%) | (100.0%) |
| Enrolled [n (%)] | 11 | 8 | 8 | 27 |
| | (100.0%) | (100.0%) | (100.0%) | (100.0%) |
| Dosed [n (%)] | 11 | 8 | 8 | 27 |
| | (100.0%) | (100.0%) | (100.0%) | (100.0%) |
| Completed the | 11 | 8 | 8 | 27 |
| Study [n (%)] | (100.0%) | (100.0%) | (100.0%) | (100.0%) |
| Discontinued | 0 | 0 | 0 | 0 |
| the Study [n (%)] | (0.0%) | (0.0%) | (0.0%) | (0.0%) |

Subjects were recruited to this study so that 16 subjects with hepatic impairment (8 subjects with mild impairment and 8 subjects with moderate impairment, per CP classification, Table 3) and 8 to 12 subjects with normal hepatic function were enrolled, with the goal of having at least 6 subjects from each hepatic impairment group and sufficient matching subjects with normal hepatic function complete the study.

Hepatic impairment was classified using the CP System, and the parameters to determine the CP class for each subject with hepatic impairment were collected at Screening and re-collected at Check-in (Day −1). If the hepatic function classification for the subject was not similar at the 2 timepoints, enrollment of the subject into a hepatic category group was based on the CP score at Screening. The CP System is presented in Table 3.

TABLE 3
Child-Pugh Assessment of Hepatic Function

| | Points Scored for Observed Findings | | |
|---|---|---|---|
| Parameter | 1 | 2 | 3 |
| Hepatic encephalopathy | 0 | 1 or 2^{b} | 3 or 4^{b} |
| grade^{a} | | | |
| Ascites^{c} | Absent | Slight | Moderate to |
| | | | Severe |
| Serum bilirubin (mg/dL) | <2 | 2 to 3 | >3 |
| Serum albumin (g/dL) | >3.5 | 2.8 to 3.5 | <2.8 |
| International normalized | <1.7 | 1.7 to 2.3 | >2.3 |
| ratio | | | |

^{a}Grade 0: normal consciousness, personality, neurological examination, or electroencephalogram.
Grade 1: restless, sleep disturbed, irritable/agitated, tremor, impaired handwriting, or 5 cycles per second (cps) waves.
Grade 2: lethargic, time-disoriented, inappropriate, asterixis, ataxia, or slow triphasic waves.
Grade 3: somnolent, stuporous, place-disoriented, hyperactive reflexes, rigidity, or slower waves.
Grade 4: unarousable coma, no personality/behavior, decerebrate, or slow 2 to 3 cps delta activity.
^{b}A subject with hepatic encephalopathy of Grade 2 or above was not admitted into the study.
^{c}Absent: No ascites was detectable by manual examination or by ultrasound investigation, if ultrasound investigation was performed.
Slight: Ascites palpation doubtful, but ascites measurable by ultrasound investigation, if performed.
Moderate: Ascites detectable by palpation and by ultrasound investigation, if performed.
Severe: Necessity of paracentesis; does not respond to medication treatment.

Each matched-control healthy subject (Group 1) was enrolled following the enrollment of a mild and/or moderate hepatic impairment subject and demographically matched by age (±10 years), body mass index (BMI) (±20%), and sex to the enrolled hepatic impairment subject(s).

Due to the potential impact of CYP2D6 genetic polymorphism on the metabolism of rivoceranib, subjects were genotyped for CYP2D6. Slow and non-CYP2D6 metabolizers were excluded from the study to ensure better population homogeneity for the small sample size. The CYP3A4 polymorphisms are rare or lack phenotypic effect; therefore, there was no need to genotype for CYP3A4.

The Safety Population consisted of all subjects who received the single dose of rivoceranib and had at least 1 postdose safety assessment.

The PK Population consisted of all subjects who received the single dose of rivoceranib and had evaluable PK data. Data was excluded from the PK summary statistics and statistical analysis if the subject had an AE of vomiting that occurred at or before 2 times median time to maximum concentration (tₘₐₓ).

The All Subjects Population consisted of all subjects who signed an ICF and had study.

### Assessments Recorded in the Database Per the Protocol.

Up to eight subjects were enrolled in each hepatic impairment group in order that at least 6 subjects per hepatic impairment group complete the study. Eight to 12 subjects were enrolled in the normal hepatic function group to ensure an adequate number of matched-control subjects. The sample size was determined without power consideration on the basis of other studies similar in design.

Table 4 summarizes CP Score by Hepatic function. The mean CP Score for the Mild hepatic impairment group was 5.3 and in the Moderate hepatic impairment group was 7.6.

TABLE 4
Summary of Child-Pugh Score (Safety Population)

| | | | |
|---|---|---|---|
| Normal | Mild | Moderate | |
| Hepatic | Hepatic | Hepatic | |
| Function | Impairment | Impairment | Overall |

Points from Child-Pugh Assessment

| | | | | |
|---|---|---|---|---|
| N | — | 16 | 16 | 32 |
| Mean (SD) | — | 5.3 (0.45) | 7.6 (0.96) | 6.4 (1.41) |
| Median | — | 5.0 | 7.0 | 6.5 |
| Min, Max | — | 5, 6 | 7, 10 | 5, 10 |

Dose determination: Enrolled patients are treated with rivoceranib a single 200 mg dose tablet of rivoceranib (containing 248 mg rivoceranib mesylate). The patients were given rivoceranib orally as a 1×200 mg tablet on Day 1 after an overnight fast (at least 10 hours).

A single dose administration was predicted to accurately describe the PK of the drug based on previous clinical studies which demonstrated that exposure of rivoceranib is time independent and dose proportional. The safety and PK assessments are standard parameters for clinical studies in drug development. Plasma sampling was timed to sufficiently estimate PK parameters of rivoceranib exposure and allow determination of PK profiles of metabolites.

A Phase 3 study (LSK-AM301) conducted for gastric cancer included administration of 700 mg rivoceranib. The slopes and 95% confidence interval (CI) indicate that the PK of rivoceranib is dose proportional between 81 and 685 mg, following single- and multiple-dose administration.

Given the known human experience for rivoceranib, this Phase 1 study evaluated the PK of 200 mg rivoceranib in subjects with normal hepatic function and stable hepatic impairment. This dose was justified by reported human data which demonstrates that 200 mg is a conservative safe dose for rivoceranib and afforded a safety window anticipating increasing exposure as hepatic impairment worsens.

Study Duration: Serial blood sample collections were obtained for the analysis of plasma concentrations of rivoceranib and metabolites M1-1, M1-2, M1-6, and M9-2. The plasma PK parameters of rivoceranib and metabolites were calculated using standard noncompartmental methods following administration of rivoceranib on Day 1.

The following PK parameter endpoints were calculated:
maximum observed plasma concentration (Cₘₐₓ), area under the plasma concentration-time curve (AUC) from time zero to infinity (AUC_{0-∞}), AUC from time zero to time of last quantifiable concentration (AUC₀₋ₜ), time of the Cmax (tmax), apparent plasma terminal elimination half-life (t_{1/2}), apparent total plasma clearance (CL/F; rivoceranib only), apparent volume of distribution during the terminal elimination phase (V_{z}/F; rivoceranib only), metabolic ratio based on AUC (MR_{AUC}; M1-1, M1-2, M1-6, and M9-2 only), metabolic ratio based on Cₘₐₓ (MR_{Cmax}; M1-1, M1-2, M1-6, and M9-2 only), and fraction of unbound drug (fu). Other noncompartmental parameters could be reported.

Safety measures for this study included AEs, incidence of laboratory abnormalities (based on hematology, clinical chemistry, coagulation, and urinalysis test results), 12-lead ECG parameters, vital sign measurements, and physical examinations.

The primary analysis planned for this study was to evaluate the PK profile of rivoceranib after a single dose in subjects with mild or moderate hepatic impairment compared to subjects with normal hepatic function. The PK profiles of rivoceranib metabolites were also evaluated. The following statistical methodology was used, based on one-to-one matching:

If an individual healthy subject was matched to 1 subject from any or all hepatic impairment groups, a paired t-test was applied to analyze the natural log-transformed primary PK parameters (Cₘₐₓ, AUC₀₋ₜ, and AUC_{0-∞}) for rivoceranib and metabolites M1-1, M1-2, M1-6, and M9-2. The paired t-test was performed separately for each hepatic impairment group and the corresponding matched healthy controls. Estimates of geometric mean ratios in primary PK parameters between each level of impaired hepatic function versus the matched healthy controls were presented along with the corresponding 90% confidence intervals (CIs) for the geometric mean ratios. The p-value assessing the difference between each impairment group and the healthy control group was presented.

Plasma concentrations and PK parameters were summarized by group using descriptive statistics (number, arithmetic mean, standard deviation, geometric mean, geometric coefficient of variation, median, minimum, and maximum). In addition, summary statistics for protein binding was tabulated by hepatic function group.

All adverse events and observed values from clinical laboratory evaluations, 12-lead ECGs, vital sign measurements, and physical examinations were listed and summarized descriptively. No inferential statistical analyses were planned for the safety data.

Study treatment details: Rivoceranib is administered as the mesylate salt of its free base, provided as a 200 mg tablet. Rivoceranib doses as provided in this example, are given as the amount of freebase rather than the mesylate salt. The freebase dosage is approximately 81% of the mesylate dosage. The formulation is the same. Referring to Rivoceranib dose strength as freebase aligns with standards for referencing total active product.

Pharmacokinetic Assessment: Serial blood sample collections were obtained for the analysis of plasma concentrations of rivoceranib and metabolites M1-1, M1-2, M1-6, and M9-2. The plasma PK parameters of rivoceranib and metabolites were calculated using standard noncompartmental methods following administration of rivoceranib on Day 1.

Results: Subjects with normal hepatic function were instructed to refrain from use of any prescription or nonprescription medications/products during the study until the follow-up phone call, unless the Investigator and/or Sponsor had given their prior consent.

For subjects with mild or moderate hepatic impairment, treatment with chronic stable medications necessary for maintaining the clinical status of the subject were permitted if prescribed by the subject's personal physician and approved by the Medical Monitor and Investigator, in consultation with the Sponsor as needed. Administration of medications were withheld for at least 2 hours predose and 4 hours postdose as clinically appropriate.

The occasional use of paracetamol/acetaminophen (<1 g/day), hormone replacement therapy, oral, implantable, transdermal, injectable, or intrauterine contraceptives were acceptable concomitant medications. The administration of any other concomitant medications during the study was prohibited without prior approval of the Investigator, unless its use was deemed necessary for treatment of an AE. Any medication taken by a subject during the course of the study and the reason for its use was documented in the source data.

All doses were administered under the supervision of suitably qualified study site staff. Immediately after dose administration, a hand and mouth check was performed to verify that the doses administered were swallowed. The assessments and their timing in relation to dose are listed in Table 5 below.

TABLE 5
Schedule of Assessments

| | | | | | | Follow-up |
|---|---|---|---|---|---|---|
| | Screening | Check- | | | Discharge | Phone Call |
| | (Days −35 | in | | | or ET^{a} | Day 7 |
| Study Procedures | to −2) | (Day −1) | Day 1 | Day 2 | Day 3 | (±2 days) |
| Informed Consent | X | | | | | |
| Admission to CRU | | X | | | | |
| Discharge from CRU | | | | | X | |
| Review | X | X | | | | |
| Inclusion/Exclusion | | | | | | |
| Criteria | | | | | | |
| Demographics | X | | | | | |
| Child-Pugh Class Score^{b} | X | X | | | | |
| CYP2D6 Genotyping^{c} | X | | | | | |
| Medical History | X | X^{d} | | | | |
| Height, Weight, and BMI | X | X^{e} | | | | |
| Drug and Alcohol Screen^{f} | X | X | | | | |
| Hepatitis and HIV Screen | X | | | | | |
| Pregnancy Test^{g} | X | X | | | X | |
| FSH Test^{h} | | | | | | |
| Physical Examinationⁱ | X | X | X | | X | |
| Clinical Laboratory | X | X | | X | X | |
| Evaluations^{j} | | | | | | |
| Twelve-lead ECG^{k} | X | X | X | | X | |
| Vital Signs^{l} | X | X | X | X | X | |
| Hemoglobin A1c test^{m} | X | | | | | |
| Rivoceranib Doseⁿ | | | X | | | |
| Primary PK Blood | | | X | X | X | |
| Samples^{o} | | | | | | |
| Unbound Drug (Protein | | | X | X | | |
| Binding) Blood | | | | | | |
| Sample^{p} | | | | | | |
| Prior and Concomitant | X | X | X | X | X | X |
| Medications^{q} | | | | | | |
| Adverse Event Inquiry^{r} | | | X | X | X | X |
| Serious AE Monitoring^{s} | X | X | X | X | X | X |

Abbreviations: AE = adverse event; BMI = body mass index; CP = Child-Pugh; CRU = Clinical Research Unit; CYP = cytochrome P450; ECG = electrocardiogram; ET = Early Termination; FSH = follicle-stimulating hormone: HIV = human immunodeficiency virus; PK = pharmacokinetic; SAE = serious adverse event.
^{a}Subjects who withdrew from the study prior to Day 3 were to undergo all Discharge tests and assessments at ET.
^{b}Subjects with hepatic impairment only. Child-Pugh (CP) scores were calculated at Screening and repeated at Check-in; hepatically-impaired subjects were assigned to groups according to CP scores at Screening and their hepatic impairment rechecked at Check-in to ensure stability of hepatic impairment and subject safety, as determined by the Investigator and Covance Medical Monitor.
^{c}CYP2D6 genotype testing was performed in order to exclude slow- and non-CYP2D6 metabolizers.
^{d}Interim medical history update only.
^{e}Weight only.
^{f}Alcohol test and drugs of abuse urine screen. The drugs of abuse urine screen (including cotinine for matched-control healthy subjects only) was performed at Screening and Check-in and the alcohol test was performed at Check-in. Results from the alcohol and drug tests were used to determine subject eligibility per the inclusion/exclusion criteria.
^{g}Female subjects only. Serum pregnancy test was performed at Screening and urine pregnancy tests performed at Check-in and Discharge (or ET). If urine test was positive, a confirmatory serum test was performed.
^{h}Postmenopausal female subjects only.
ⁱA complete physical examination was performed at Screening and Discharge (or ET). An abbreviated physical examination (general appearance, skin, thorax/lungs, cardiovascular system, and abdomen) was performed at Check-in and 1 hour postdose on Day 1.
^{j}Clinical chemistry (fasted 10 hours), hematology, coagulation parameters, and urinalysis was performed at Screening, Check-in, 24 hours postdose (Day 2), and at Clinic Discharge (Day 3) or ET.
^{k}Single 12-lead ECGs were collected at Screening, Check-in, and at Discharge/ET and triplicate 12-lead ECGs were collected on Day 1 at predose and 4 hours (±10 minutes) postdose. The ECGs were collected after the subject had rested in the supine position for at least 5 minutes, and obtained prior to and as close as possible to the scheduled blood draws.
^{l}Vital sign measurements (oral temperature, supine blood pressure, and pulse rate) were obtained at Screening and Check-in; at predose, 2 hours (±10 minutes), and 4 hours (±10 minutes) postdose on Day 1; on Day 2 (approximately 24 hours postdose); and on Day 3 (prior to Discharge). Vital sign measurements were carried out prior to and as close as possible to having blood drawn. Blood pressure and pulse rate were measured using the same arm for each reading after the subject has been supine for at least 5 minutes.
^{m}Hemoglobin A1c test performed at Screening for subjects with hepatic impairment only.
ⁿDose administration was to be given during the morning of Day 1.
^{o}Primary PK blood samples were collected predose, and at 0.5, 1, 2, 3, 4, 6, 8, 12, 16, 24, 36, and 48 hours postdose. The allowed sampling window for PK blood samples was as follows: within 15 minutes prior to dosing for the predose sample timepoint; ±5 minutes for sampling timepoints within the first 12 hours; ±30 minutes for sampling timepoints >12 hours ≤36 hours; and ±60 minutes for the sampling timepoint at 48 hours.
^{p}For assessment of unbound plasma concentrations of rivoceranib, a blood sample was collected at 4 and 24 hours postdose.
^{q}Prior and concomitant medication administration were recorded beginning at informed consent. In addition, all Investigator-approved medications taken by a subject within 30 days prior to study drug administration for prescription medications, and 7 days prior to study drug administration for nonprescription medications, were recorded on the subject's electronic Case Report Form.
^{r}Adverse events (AEs) were recorded beginning at dose administration.
^{s}Serious adverse events (SAEs) were recorded beginning at informed consent.

Blood samples were collected at specific times during the study (Table 3) for the measurement of plasma concentrations of rivoceranib and metabolites M1-1, M1-2, M1-6, and M9-2.

The PK analysis was conducted by Covance Early Clinical Biometrics using WinNonlin Version 8.1.

The PK parameters presented in Table 6 below were determined from the plasma concentrations of rivoceranib and metabolites M1-1, M1-2, M1-6, and M9-2 using noncompartmental procedures.

TABLE 6
Pharmacokinetic Parameters Determined for Rivoceranib and Metabolites

| Parameter | Definition |
|---|---|
| Cₘₐₓ | maximum observed plasma concentration |
| AUC₀₋ₜ | area under the concentration-time curve from time 0 to the time of last |
| | quantifiable concentration |
| AUC_{0-∞} | arca under the concentration-time curve from time 0 to infinity |
| tₘₐₓ | time of maximum observed plasma concentration |
| t_{1/2} | apparent plasma terminal elimination half-life |
| CL/F | apparent total plasma clearance (rivoceranib only) |
| V_{z}/F | apparent volume of distribution during the terminal elimination phase |
| | (rivoceranib only) |
| MR_{Cmax} | metabolic ratio for Cₘₐₓ (M1-1, M1-2, M1-6, and M9-2 only) |
| MR_{AUC} | metabolic ratio for AUC (M1-1, M1-2, M1-6, and M9-2 only) |
| fu | fraction of unbound drug (rivoceranib only) |

Blood samples were collected at specific times during the study (Table 3) for the measurement of fraction unbound of rivoceranib (protein binding). The analysis for fraction unbound of rivoceranib was performed by a laboratory using qualified analytical procedures.

The condition of each subject was monitored throughout the study. Any AEs and remedial actions required were recorded. The nature, time of onset, duration, and severity were documented, together with the Investigator's opinion of the relationship to drug administration. Any clinically significant abnormalities found during the course of the study were followed up until they returned to normal or could be clinically explained.

There were no instances of AEs, concomitant medications, or deviations which impact the PK interpretation. There were ten samples that were collected outside the 10% of the collection time window. The values are included in the PK analysis using actual time, however, have been excluded from summary statistics and mean concentration-time figures.

In 25 subjects % AUCₑₓₜᵣₐₚ was greater than 20%. This was mostly for metabolites M1-1, M1-2, and M9-2. In these instances, AUC_{0-∞} as well as parameters derived from AUC_{0-∞} (CL/F and V_{z}/F for rivoceranib, and MR_{AUC} for metabolite M1-1, M1-2, M1-6, and M9-2), have been excluded from summary statistics.

Eighty profiles mostly for metabolites M1-1, M1-2, and M9-2 across all 27 subjects are flagged for the regression being determined over a period of <2 times resultant t_{1/2}, with most flags for metabolites M1-1, M1-2, and M9-2. For all these profiles, the R² adjusted values are >0.7; the values are considered to be robust. Therefore, these values are flagged and footnoted, but included in summary statistics.

Summaries of the plasma concentrations of rivoceranib are presented in Table 7. The plasma concentration versus time profiles for rivoceranib in normal hepatic function, and mild and moderate hepatic impairment subjects were characterized by a rapid absorption phase.

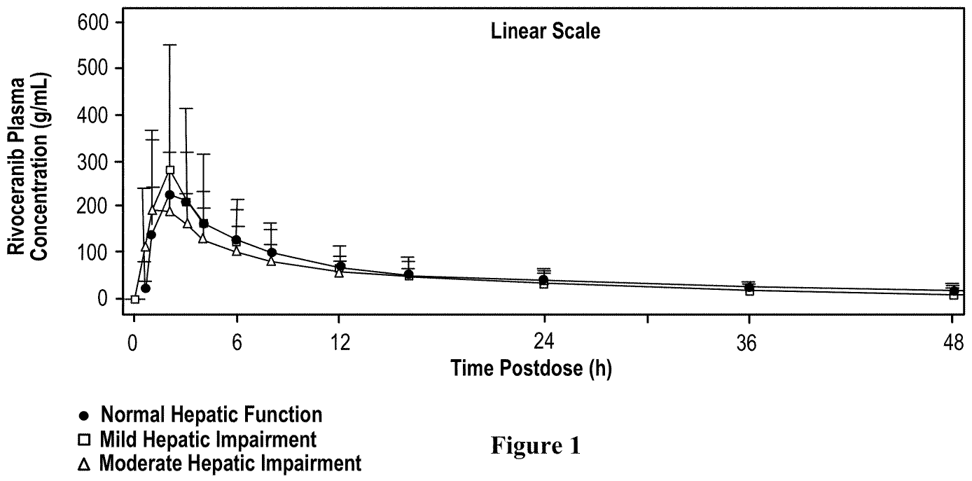

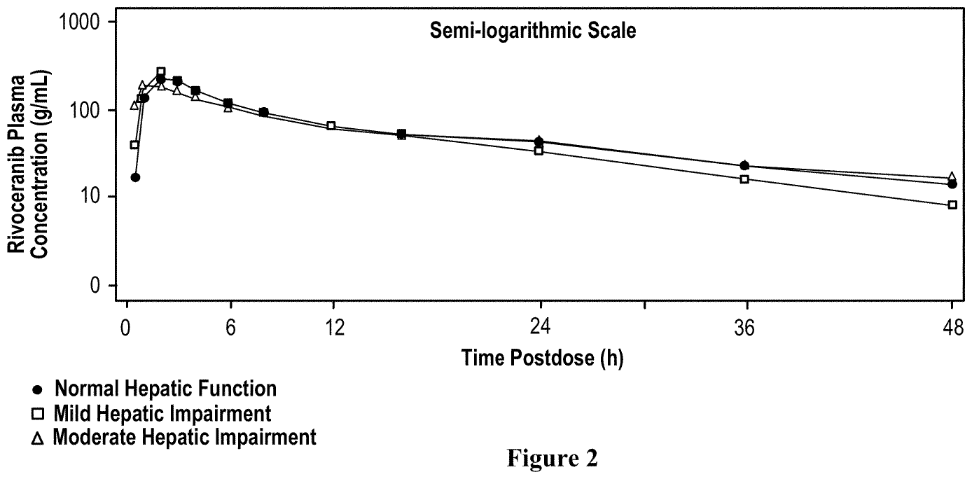

Arithmetic mean plasma concentration-time profiles of rivoceranib, following administration of rivoceranib in normal hepatic function, and mild and moderate hepatic impairment subjects are presented in FIG. 1 and FIG. 2.

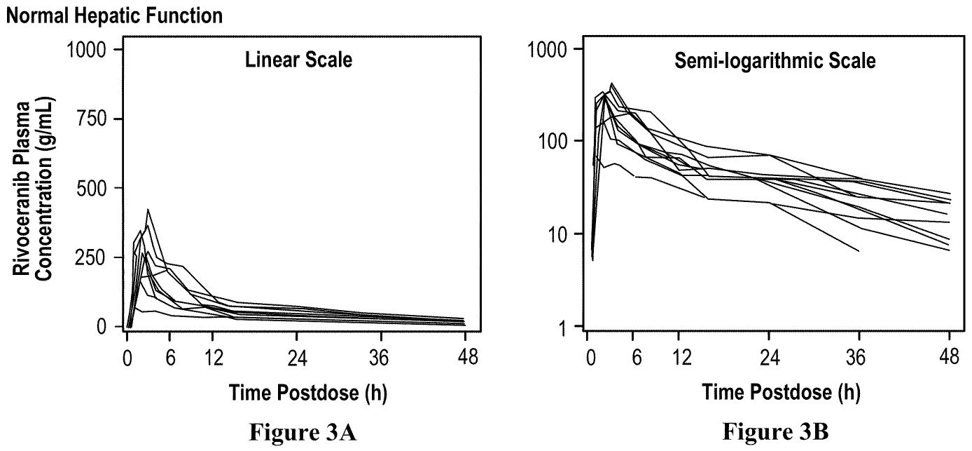

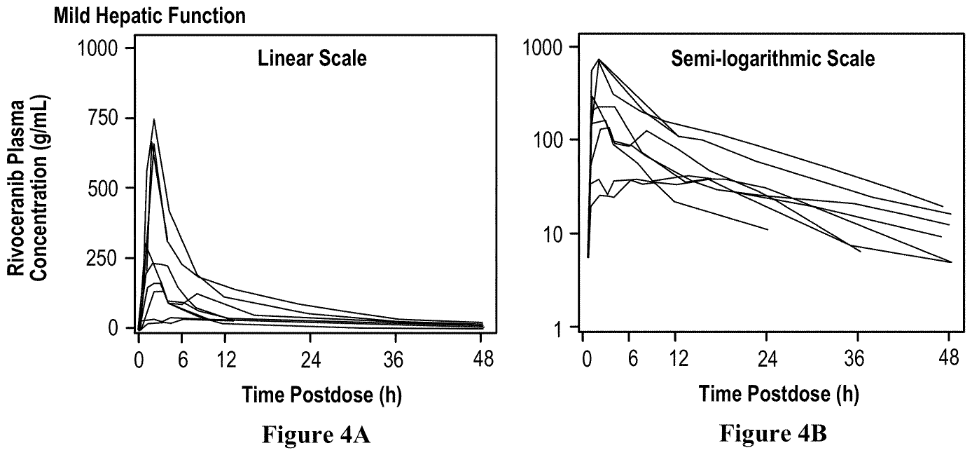

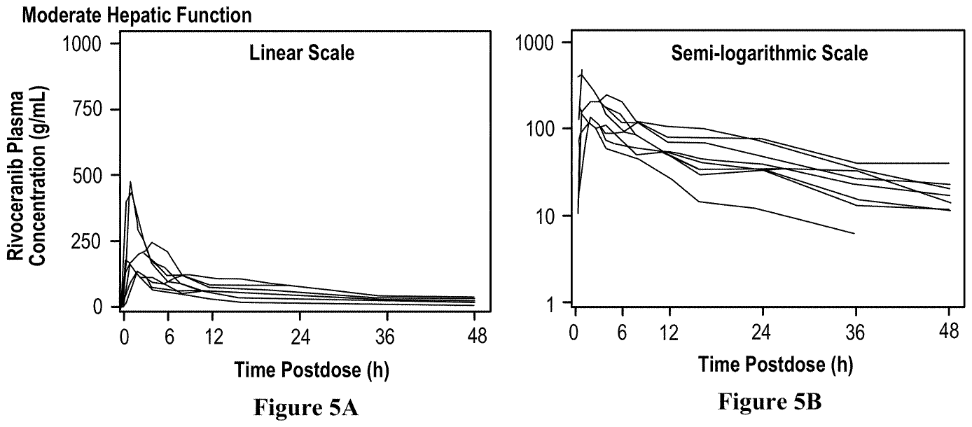

Overlaying Individual Pharmacokinetic Concentration-time Profiles for rivoceranib, following administration of rivoceranib in normal hepatic function, and mild and moderate hepatic impairment subjects showing spread of concentrations in subjects are presented in FIG. 3, FIG. 4, and FIG. 5, respectively.

The summary of unbound PK concentrations and fraction unbound rivoceranib is presented in Table 8 below. Mean±standard deviation (SD) fraction unbound at 4 hours appeared to be higher for moderate hepatic impairment subjects (4.25±2.204%) compared to the normal hepatic function and mild hepatic impairment subjects (2.65±1.189% and 2.39±0.639%, respectively). Mean fraction unbound was similar at 24 hours in all three groups.

TABLE 7
Summary of Plasma Concentrations

| Hepatic | | | | Arithmetic | Arithmetic | | Geometric | | | | |
|---|---|---|---|---|---|---|---|---|---|---|---|
| Function | | | | Mean | SD | Arithmetic | Mean | Geometric | Median | Min | Max |
| Group | Analyte | Timepoint | N | (ng/mL) | (ng/mL) | CV (%) | (ng/mL) | CV (%) | (ng/mL) | (ng/mL) | (ng/mL) |
| Normal | Rivoceranib | Predose | 11 | 0.00 | 0.000 | — | — | — | 0.00 | 0.0 | 0.0 |
| Hepatic | | 0.5 h | 11 | 17.66 | 24.780 | 140.3% | 15.22 | 131.1% | 6.80 | 0.0 | 74.6 |
| Function | | 1 h | 11 | 136.01 | 107.781 | 79.2% | 92.93 | 135.6% | 74.40 | 11.0 | 306.0 |
| | | 2 h | 11 | 226.43 | 90.784 | 40.1% | 203.86 | 58.1% | 236.00 | 52.7 | 350.0 |
| | | 3 h | 11 | 215.71 | 103.194 | 47.8% | 191.75 | 58.3% | 199.00 | 57.8 | 417.0 |
| | | 4 h | 11 | 163.86 | 71.805 | 43.8% | 149.14 | 49.9% | 147.00 | 57.5 | 305.0 |
| | | 6 h | 11 | 130.99 | 62.682 | 47.9% | 116.65 | 56.2% | 106.00 | 42.1 | 224.0 |
| | | 8 h | 11 | 99.38 | 50.347 | 50.7% | 89.48 | 50.1% | 81.00 | 41.0 | 216.0 |
| | | 12 h | 11 | 67.83 | 26.443 | 39.0% | 63.25 | 41.2% | 61.90 | 31.5 | 114.0 |
| | | 16 h | 11 | 48.84 | 18.327 | 37.5% | 45.71 | 40.6% | 49.70 | 23.6 | 88.9 |
| | | 24 h | 11 | 43.23 | 16.747 | 38.7% | 40.38 | 40.7% | 43.00 | 21.7 | 74.1 |
| | | 36 h | 11 | 23.91 | 10.925 | 45.7% | 21.26 | 58.8% | 24.20 | 6.6 | 40.3 |
| | | 48 h | 11 | 14.88 | 8.401 | 56.5% | 14.78 | 53.1% | 15.90 | 0.0 | 27.4 |
| Mild | Rivoceranib | Predose | 8 | 0.00 | 0.000 | — | — | — | 0.00 | 0.0 | 0.0 |
| Hepatic | | 0.5 h | 8 | 41.46 | 41.305 | 99.6% | 29.45 | 178.1% | 30.25 | 0.0 | 116.0 |
| Impairment | | 1 h | 8 | 178.40 | 168.577 | 94.5% | 111.83 | 158.8% | 149.00 | 20.2 | 524.0 |
| | | 2 h | 8 | 278.66 | 274.240 | 98.4% | 165.39 | 179.0% | 194.00 | 25.9 | 743.0 |
| | | 3 h | 8 | 219.64 | 197.039 | 89.7% | 138.09 | 166.9% | 155.00 | 25.7 | 574.0 |
| | | 4 h | 8 | 164.38 | 146.050 | 88.9% | 111.52 | 129.0% | 95.85 | 24.8 | 438.0 |
| | | 6 h | 8 | 122.01 | 93.835 | 76.9% | 95.41 | 86.5% | 91.30 | 38.5 | 297.0 |
| | | 8 h | 8 | 96.01 | 66.249 | 69.0% | 77.78 | 78.7% | 68.55 | 33.8 | 195.0 |
| | | 12 h | 8 | 66.58 | 45.438 | 68.3% | 54.88 | 73.6% | 43.85 | 21.8 | 150.0 |
| | | 16 h | 8 | 54.26 | 36.857 | 67.9% | 45.27 | 70.2% | 39.65 | 17.2 | 124.0 |
| | | 24 h | 8 | 34.48 | 23.315 | 67.6% | 29.00 | 67.9% | 26.00 | 11.2 | 82.7 |
| | | 36 h | 8 | 16.06 | 12.888 | 80.2% | 15.20 | 75.8% | 13.75 | 0.0 | 40.0 |
| | | 48 h | 8 | 8.50 | 7.203 | 84.8% | 9.97 | 62.4% | 7.26 | 0.0 | 19.3 |
| Moderate | Rivoceranib | Predose | 8 | 0.00 | 0.000 | — | — | — | 0.00 | 0.0 | 10.0 |
| Hepatic | | 0.5 h | 8 | 115.11 | 129.954 | 112.9% | 57.12 | 247.3% | 92.25 | 10.3 | 398.0 |
| Impairment | | 1 h | 8 | 195.30 | 166.928 | 85.5% | 140.20 | 110.4% | 154.50 | 41.4 | 478.0 |
| | | 2 h | 8 | 190.38 | 77.306 | 40.6% | 178.06 | 39.8% | 169.50 | 117.0 | 326.0 |
| | | 3 h | 8 | 165.73 | 65.862 | 39.7% | 153.92 | 43.6% | 161.50 | 88.8 | 255.0 |
| | | 4 h | 8 | 134.24 | 63.253 | 47.1% | 121.21 | 52.4% | 132.00 | 58.8 | 247.0 |
| | | 6 h | 8 | 106.20 | 50.678 | 47.7% | 96.85 | 47.8% | 93.80 | 51.2 | 206.0 |
| | | 8 h | 8 | 85.46 | 30.661 | 35.9% | 80.32 | 40.1% | 85.00 | 46.1 | 122.0 |
| | | 12 h | 8 | 62.34 | 24.211 | 38.8% | 58.24 | 41.9% | 54.25 | 27.9 | 107.0 |
| | | 16 h | 8 | 52.63 | 28.950 | 55.0% | 45.33 | 67.5% | 43.40 | 14.9 | 102.0 |
| | | 24 h | 8 | 44.26 | 22.331 | 50.5% | 38.74 | 65.0% | 36.55 | 11.7 | 78.7 |
| | | 36 h | 8 | 24.00 | 11.658 | 48.6% | 20.85 | 68.7% | 24.85 | 6.2 | 39.8 |
| | | 48 h | 8 | 17.36 | 11.759 | 67.7% | 18.08 | 46.9% | 15.50 | 0.0 | 40.7 |

TABLE 8
Summary of Unbound Pharmacokinetic Concentrations and Fraction Unbound

| Hepatic | | | | Arithmetic | Arithmetic | | Geometric | | | | |
|---|---|---|---|---|---|---|---|---|---|---|---|
| Function | | | | Mean | SD | Arithmetic | Mean | Geometric | Median | Min | Max |
| Group | Parameter | Timepoint | N | (ng/mL) | (ng/mL) | CV (%) | (ng/mL) | CV (%) | (ng/mL) | (ng/mL) | (ng/mL) |
| Normal | Unbound | 4 h | 11 | 4.44 | 2.987 | 67.3% | 3.62 | 78.6% | 3.76 | 1.0 | 11.8 |
| Hepatic | Concentration | 24 h | 11 | 0.73 | 0.300 | 40.8% | 0.68 | 44.3% | 0.69 | 0.3 | 1.3 |
| Function | (ng/mL) | | | | | | | | | | |
| Mild | Unbound | 4 h | 8 | 3.78 | 3.408 | 90.3% | 2.59 | 124.0% | 2.37 | 0.7 | 10.5 |
| Hepatic | Concentration | 24 h | 8 | 0.63 | 0.286 | 45.5% | 0.55 | 64.0% | 0.65 | 0.2 | 1.1 |
| Impairment | (ng/mL) | | | | | | | | | | |
| Moderate | Unbound | 4 h | 8 | 4.73 | 1.563 | 33.1% | 4.52 | 31.8% | 4.15 | 3.3 | 7.7 |
| Hepatic | Concentration | 24 h | 8 | 1.07 | 0.825 | 77.1% | 0.82 | 98.5% | 0.82 | 0.2 | 2.5 |
| Impairment | (ng/mL) | | | | | | | | | | |
| Normal | Fraction | 4 h | 11 | 2.65 | 1.189 | 44.9% | 2.43 | 44.7% | 2.12 | 1.6 | 4.7 |
| Hepatic | Unbound (%) | 24 h | 11 | 1.72 | 0.396 | 23.0% | 1.68 | 22.6% | 1.55 | 1.3 | 2.5 |
| Function | | | | | | | | | | | |
| Mild | Fraction | 4 h | 8 | 2.39 | 0.637 | 26.6% | 2.32 | 26.9% | 2.32 | 1.6 | 3.4 |
| Hepatic | Unbound (%) | 24 h | 8 | 2.01 | 0.689 | 34.3% | 1.91 | 33.8% | 1.74 | 1.3 | 3.1 |
| Impairment | | | | | | | | | | | |
| Moderate | Fraction | 4 h | 8 | 4.25 | 2.204 | 51.9% | 3.73 | 59.9% | 4.19 | 2.0 | 7.6 |
| Hepatic | Unbound (%) | 24 h | 8 | 2.20 | 0.699 | 31.8% | 2.10 | 33.1% | 2.10 | 1.3 | 3.4 |
| Impairment | | | | | | | | | | | |

The summary statistics of PK parameters for rivoceranib in normal hepatic function, and mild and moderate hepatic impairment groups are presented in Table 9 below. The plasma concentration versus time profiles for rivoceranib in normal hepatic function subjects as well as mild and moderate hepatic impairment subjects were characterized by a rapid absorption phase. Geometric mean Cₘₐₓ was the highest in the normal hepatic function subjects (249.69 ng/mL) followed by moderate hepatic impairment (210.05 ng/ml) and the lowest in the mild impairment subjects (182.22 ng/ml). Geometric mean AUC_{0-∞} was the highest in the normal hepatic function subjects (3051.30 h*ng/mL) followed by moderate hepatic impairment (2458.98 h*ng/mL) and the lowest in the moderate impairment subjects (2308.6 h*ng/mL).

Between-subject variability for rivoceranib in normal function, mild hepatic impairment and moderate hepatic impairment group based on AUC_{0-∞} (geometric CV, 31.5%, 77.0%, and 49.1% respectively) and Cₘₐₓ (geometric CV %, 50.8%, 158.6%, and 56.7%, respectively) Median tₘₐₓ (min-max) was similar following administration of rivoceranib in normal hepatic function, mild hepatic impairment and moderate hepatic impairment group (2.0 [1-6], 2.5 [1-12] and 2.0 [1-4] hours, respectively). After reaching Cₘₐₓ, plasma concentrations of rivoceranib appeared to decline in a biphasic manner. The arithmetic mean±SD t_{1/2} was similar in normal, mild and moderate hepatic impairment group (17.907±8.6025, 13.304±5.5814 and 18.747±3.9656 hours, respectively).

Pharmacokinetic parameters for M1-1, M1-2, M1-6, and M9-2 are summarized here. Median tₘₐₓ (min-max) of all metabolites was relatively unchanged between three groups. Similar to that of rivoceranib, exposure and Cₘₐₓ was unchanged of all the metabolites in normal hepatic function, mild impairment and moderate hepatic impairment groups.

TABLE 9
Summary of Pharmacokinetic Parameters

| | | Hepatic | | | | | | | | | | |
|---|---|---|---|---|---|---|---|---|---|---|---|---|
| | | Function | | 90% | Arithmetic | Arithmetic | Arithmetic | Geometric | Geometric | | | |
| Analyte | Parameter | Group | N | C.I. | Mean | SD | CV (%) | Mean | CV (%) | Median | Min | Max |
| Rivoceranib | AUC_{0-∞} | Normal | 9 | (2598.40, | 3178.61 | 936.044 | 29.4% | 3051.30 | 31.5% | 2878.14 | 1948.2 | 4529.9 |
| | (h*ng/mL) | Hepatic | | 3758.81) | | | | | | | | |
| | | Function | | | | | | | | | | |
| | | Mild Hepatic | 7 | (1341.38, | 2842.68 | 2044.111 | 71.9% | 2308.66 | 77.0% | 2140.79 | 1066.4 | 5817.2 |
| | | Impairment | | 4343.99) | | | | | | | | |
| | | Moderate | 6 | (1777.25, | 2664.21 | 1078.194 | 40.5% | 2458.98 | 49.1% | 2589.30 | 1065.3 | 4426.4 |
| | | Hepatic | | 3551.18) | | | | | | | | |
| | | Impairment | | | | | | | | | | |
| | AUC₀₋ₜ | Normal | 11 | (2242.58, | 2692.35 | 823.035 | 30.6% | 2560.83 | 35.9% | 2778.08 | 1207.3 | 3845.7 |
| | (h*ng/mL) | Hepatic | | 3142.12) | | | | | | | | |
| | | Function | | | | | | | | | | |
| | | Mild Hepatic | 8 | (1311.18, | 2554.52 | 1856.187 | 72.7% | 2074.79 | 75.4% | 1935.05 | 976.0 | 5531.5 |
| | | Impairment | | 3797.86) | | | | | | | | |
| | | Moderate | 8 | (1872.64, | 2594.58 | 1077.794 | 41.5% | 2377.29 | 50.0% | 2448.73 | 926.2 | 4195.4 |
| | | Hepatic | | 3316.52) | | | | | | | | |
| | | Impairment | | | | | | | | | | |
| | Cₘₐₓ | Normal | 11 | (218.69, | 271.40 | 96.457 | 35.5% | 249.69 | 50.8% | 270.00 | 74.4 | 417.0 |
| | (ng/mL) | Hepatic | | 324.11) | | | | | | | | |
| | | Function | | | | | | | | | | |
| | | Mild Hepatic | 8 | (109.27, | 290.80 | 271.011 | 93.2% | 182.22 | 158.6% | 201.50 | 38.9 | 743.0 |
| | | Impairment | | 472.33) | | | | | | | | |
| | | Moderate | 8 | (146.05, | 239.38 | 139.325 | 58.2% | 210.05 | 56.7% | 180.00 | 128.0 | 478.0 |
| | | Hepatic | | 332.70) | | | | | | | | |
| | | Impairment | | | | | | | | | | |
| | tₘₐₓ (h) | Normal | 11 | (1.71, | 2.5 | 1.37 | 55.8% | 2.2 | 54.3% | 2.0 | 1 | 6 |
| | | Hepatic | | 3.20) | | | | | | | | |
| | | Function | | | | | | | | | | |
| | | Mild Hepatic | 8 | (1.28, | 3.6 | 3.50 | 96.6% | 2.8 | 82.8% | 2.5 | 1 | 12 |
| | | Impairment | | 5.97) | | | | | | | | |
| | | Moderate | 8 | (1.29, | 2.0 | 1.07 | 53.4% | 1.8 | 57.0% | 2.0 | 1 | 4 |
| | | Hepatic | | 2.72) | | | | | | | | |
| | | Impairment | | | | | | | | | | |
| | t_{1/2} (h) | Normal | 11 | (13.21, | 17.907 | 8.6025 | 48.0% | 16.260 | 47.9% | 16.942 | 9.01 | 35.66 |
| | | Hepatic | | 22.61) | | | | | | | | |
| | | Function | | | | | | | | | | |
| | | Mild Hepatic | 8 | (9.57, | 13.304 | 5.5814 | 42.0% | 12.402 | 41.2% | 12.340 | 6.61 | 24.76 |
| | | Impairment | | 17.04) | | | | | | | | |
| | | Moderate | 8 | (16.09, | 18.747 | 3.9656 | 21.2% | 18.391 | 21.1% | 18.185 | 13.47 | 25.91 |
| | | Hepatic | | 21.40) | | | | | | | | |
| | | Impairment | | | | | | | | | | |
| | CL/F (L/h) | Normal | 9 | (55.08, | 68.404 | 21.4922 | 31.4% | 65.546 | 31.5% | 69.489 | 44.15 | 102.66 |
| | | Hepatic | | 81.73) | | | | | | | | |
| | | Function | | | | | | | | | | |
| | | Mild Hepatic | 7 | (60.37, | 103.183 | 58.2890 | 56.5% | 86.630 | 77.0% | 93.423 | 34.38 | 187.55 |
| | | Impairment | | 145.99) | | | | | | | | |
| | | Moderate | 6 | (48.95, | 89.904 | 49.7849 | 55.4% | 81.335 | 49.1% | 77.251 | 45.18 | 187.74 |
| | | Hepatic | | 130.86) | | | | | | | | |
| | | Impairment | | | | | | | | | | |
| | V_{Z}/F (L) | Normal | 9 | (1151.32, | 1344.68 | 311.942 | 23.2% | 1314.68 | 22.5% | 1192.68 | 1037.7 | 1878.6 |
| | | Hepatic | | 1538.04) | | | | | | | | |
| | | Function | | | | | | | | | | |
| | | Mild Hepatic | 7 | (840.73, | 1830.59 | 1347.738 | 73.6% | 1404.24 | 95.6% | 1102.93 | 555.4 | 3685.0 |
| | | Impairment | | 2820.44) | | | | | | | | |
| | | Moderate | 6 | (1319.00, | 2247.06 | 1128.157 | 50.2% | 2017.14 | 55.9% | 2085.09 | 878.0 | 4213.4 |
| | | Hepatic | | 3175.13) | | | | | | | | |
| | | Impairment | | | | | | | | | | |

Statistical Analysis of Pharmacokinetics: The statistical analysis of PK parameters for rivoceranib in normal hepatic function, and mild and moderate hepatic impairment groups are presented in Table 10 below. The ratio of rivoceranib Cₘₐₓ GLSM was 60.1% between mild hepatic impairment subjects and subjects with normal function, suggesting that bioavailability might be decreased in the mild hepatic impairment subjects but could not be confirmed as 90% CI for the ratio for GLSM (29.8%-121.2%) fall outside 80%-125% boundary. Also, the GLSM ratio for AUC_{0-∞} and AUC₀₋ₜ between mild hepatic impairment versus normal hepatic function was 83.4% and 73.5%, respectively. Indicating that rivoceranib exposure decreased in mild hepatic impairment subjects but could not be confirmed as 90% CI of ratio of GLSM for AUC_{0-∞} and AUC₀₋ₜ fell outside the acceptable 80%-125% acceptable boundary. Similarly, the ratio of rivoceranib Cₘₐₓ GLSM was 84.9% between moderate hepatic impairment subjects and subjects with normal function, suggesting that bioavailability might be decreased in the moderate hepatic impairment subjects but could not be confirmed as 90% CI for the ratio for GLSM (45.7%-157.8%) fall outside 80%-125% boundary. Also, the GLSM ratio for AUC_{0-∞} and AUC₀₋ₜ between moderate hepatic impairment vs normal hepatic function was 81.3% and 93.2% respectively. Indicating that rivoceranib exposure decreased slightly in moderate hepatic impairment subjects but could not be confirmed as 90% CI of ratio of GLSM for AUC_{0-∞} and AUC₀₋ₜ fell outside the acceptable 80%-125% acceptable boundary.

TABLE 10
Statistical Analysis of Pharmacokinetic Parameters

| | | | 90% C.I. for the Ratio | | | | | | |
|---|---|---|---|---|---|---|---|---|---|
| | | | | | | Ratio of | | | |
| | | | | | | Geometric | | | |
| | | | | | | Least | | | |
| Geometric Least Squares Means | | | | | | Squares | | | |
| Analyte | Parameter | Comparison | N | Test | Reference | Mean | Lower | Upper | P-value |
| Rivoceranib | AUC_{0-∞} | Mild vs | 6 | 2519 | 3020 | 83.4 | 45.0 | 154.8 | 0.5802 |
| | (h*ng/mL) | Normal | | | | | | | |
| | | Moderate vs | 5 | 2428 | 2985 | 81.3 | 42.2 | 156.7 | 0.5387 |
| | | Normal | | | | | | | |
| | AUC₀₋ₜ | Mild vs | 8 | 2075 | 2821 | 73.5 | 46.3 | 116.9 | 0.2497 |
| | (h*ng/mL) | Normal | | | | | | | |
| | | Moderate vs | 8 | 2377 | 2550 | 93.2 | 57.0 | 152.4 | 0.7942 |
| | | Normal | | | | | | | |
| | Cₘₐₓ (ng/mL) | Mild vs | 8 | 182.2 | 303.4 | 60.1 | 29.8 | 121.2 | 0.2114 |
| | | Normal | | | | | | | |
| | | Moderate vs | 8 | 210.1 | 247.4 | 84.9 | 45.7 | 157.8 | 0.6321 |
| | | Normal | | | | | | | |

Safety Summary: Rivoceranib 200 mg single dose (freebase) was well tolerated in healthy subjects as well as subjects with mild and moderate hepatic impairment. AEs data were unremarkable in all groups. There were no clinically significant laboratory, ECG, or physical examinations abnormalities as determined by the investigator. There were a total of two treatment-emergent adverse events (TEAEs) in 2 (7.4%) subjects, both in the Mild hepatic impairment group: moderate diarrhea (considered possibly related to treatment) and mild infusion site extravasation (unrelated to treatment). One TEAE of these was considered related to study treatment. No treatment emergent serious adverse events (SAEs), TEAEs leading to discontinuation, or deaths were reported in this study.

Conclusions: Arithmetic mean fraction unbound at 4 hours appeared to be higher for moderate hepatic impairment subjects with large variability (4.25±2.204%) compared to the normal hepatic function and mild hepatic impairment subjects (2.65±1.189% and 2.39±0.637%, respectively). Arithmetic mean fraction unbound was similar in all three groups at 24 hours ranging from 1.72±0.396-2.20±0.699%.

After single oral dose of 200 mg rivoceranib, AUC0-∞, AUC0-t, and Cmax of rivoceranib were lowest for mild hepatic impairment subjects followed by moderate hepatic impairment subjects and normal hepatic function subjects with large inter-subject variability (geometric CV % ranging from 31.5%-158.6%). However, effect of hepatic impairment could not be confirmed as 90% CI for the GLSM ratio for rivoceranib Cmax and AUCs extended beyond 80%-125% boundary between the subjects with hepatic impairment and subjects with normal hepatic function. Due to large variability in subjects, 90% CI for the GLSM ratio for rivoceranib Cmax and AUCs extended beyond 80%-125% boundary of metabolites M1-1, M1-2, M1-6 and M9-2 effect of mild or moderate hepatic impairment and normal hepatic function subjects could not be confirmed. CL/F was lowest in normal hepatic function subjects followed by moderate hepatic impairment subjects then mild hepatic impairment subjects. The small patient size in each group could explain high variability observed.

Rivoceranib 200 mg single dose (freebase) was well tolerated in healthy subjects as well as subjects with mild and moderate hepatic impairment. AEs data were unremarkable in all groups. There were no clinically significant laboratory, ECG, or physical examinations abnormalities as determined by the investigator.

### OTHER EMBODIMENTS

While various embodiments of the present disclosure have been shown and described herein, such embodiments are provided by way of example only. Numerous variations, changes, and substitutions will now occur to those skilled in the art without departing from the inventions recited in the following claims. It should be understood that various alternatives to the embodiments of the disclosure can be employed in practice. It is intended that the following claims define the scope of the invention and that methods and structures within the scope of the claims and their equivalents be covered thereby.

What is claimed is:

1. A method of treating cancer in a subject with mild or moderate hepatic impairment, the method comprising:
administering rivoceranib or a pharmaceutically acceptable salt thereof to the subject with mild or moderate hepatic impairment, wherein rivoceranib or a pharmaceutically acceptable salt thereof is administered in a total daily amount in a range of about 100 mg to about 700 mg.

2. The method of claim 1, wherein the pharmaceutically acceptable salt of rivoceranib comprises rivoceranib mesylate salt.

3. The method of claim 1 or claim 2, wherein the total daily amount of rivoceranib or a pharmaceutically acceptable salt thereof is about 100 mg, about 200 mg, about 300 mg, about 400 mg, about 500 mg, about 600 mg, or about 700 mg.

4. The method of any one of claims 1-3, wherein the total daily amount of rivoceranib or a pharmaceutically acceptable salt thereof is at least about 200 mg.

5. The method of any one of claims 1-4, wherein rivoceranib or a pharmaceutically acceptable salt thereof is administered orally.

6. The method of any one of claims 1-5, wherein rivoceranib or a pharmaceutically acceptable salt thereof is administered in tablet form.

7. The method of any one of claims 1-6, wherein rivoceranib or a pharmaceutically acceptable salt thereof is administered once daily.

8. The method of any one of claims 1-7, wherein the subject has mild hepatic impairment.

9. The method of any one of claims 1-7, wherein the subject has moderate hepatic impairment.

10. The method of any one of claims 1-6, wherein the subject has a Child-Pugh Score of 5-6 or 7-9.

11. The method of any one of claims 1-10, wherein the cancer is selected from Acanthoma, Acinic cell carcinoma, Acoustic neuroma, Acral lentiginous melanoma, Acrospiroma, Acute eosinophilic leukemia, Acute lymphoblastic leukemia, Acute megakaryoblastic leukemia, Acute monocytic leukemia, Acute myeloblastic leukemia with maturation, Acute myeloid dendritic cell leukemia, Acute myeloid leukemia, Acute promyelocytic leukemia, Adamantinoma, Adenocarcinoma, Adenoid cystic carcinoma, Adenoma, Adenomatoid odontogenic tumor, Adrenocortical carcinoma, Adult T-cell leukemia, Aggressive NK-cell leukemia, AIDS-Related Cancers, AIDS-related lymphoma, Alveolar soft part sarcoma, Ameloblastic fibroma, Anal cancer, Anaplastic large cell lymphoma, Anaplastic thyroid cancer, Angioimmunoblastic T-cell lymphoma, Angiomyolipoma, Angiosarcoma, Appendix cancer, Astrocytoma, Atypical teratoid rhabdoid tumor, Basal cell carcinoma, Basal-like carcinoma, B-cell leukemia, B-cell lymphoma, Bellini duct carcinoma, Biliary tract cancer, Bladder cancer, Blastoma, Bone Cancer, Bone tumor, Brain Stem Glioma, Brain Tumor, Breast Cancer, Brenner tumor, Bronchial Tumor, Bronchioloalveolar carcinoma, Brown tumor, Burkitt's lymphoma, Cancer of Unknown Primary Site, Carcinoid Tumor, Carcinoma, Carcinoma in situ, Carcinoma of the penis, Carcinoma of Unknown Primary Site, Carcinosarcoma, Castleman's Disease, Central Nervous System Embryonal Tumor, Cerebellar Astrocytoma, Cerebral Astrocytoma, Cervical Cancer, Cholangiocarcinoma, Chondroma, Chondrosarcoma, Chordoma, Choriocarcinoma, Choroid plexus papilloma, Chronic Lymphocytic Leukemia, Chronic monocytic leukemia, Chronic myelogenous leukemia, Chronic Myeloproliferative Disorder, Chronic neutrophilic leukemia, Clear-cell tumor, Colon Cancer, Colorectal cancer, Craniopharyngioma, Cutaneous T-cell lymphoma, Degos disease, Dermatofibrosarcoma protuberans, Dermoid cyst, Desmoplastic small round cell tumor, Diffuse large B cell lymphoma, Dysembryoplastic neuroepithelial tumor, Embryonal carcinoma, Endodermal sinus tumor, Endometrial cancer, Endometrial Uterine Cancer, Endometrioid tumor, Enteropathy-associated T-cell lymphoma, Ependymoblastoma, Ependymoma, Epithelioid sarcoma, Erythroleukemia, Esophageal cancer, Esthesioneuroblastoma, Ewing's sarcoma, Extracranial Germ Cell Tumor, Extragonadal Germ Cell Tumor, Extrahepatic Bile Duct Cancer, Extramammary Paget's disease, Fallopian tube cancer, Fetus in fetu, Fibroma, Fibrosarcoma, Follicular lymphoma, Follicular thyroid cancer, Gallbladder Cancer, Ganglioglioma, Ganglioneuroma, Gastric Cancer, Gastric lymphoma, Gastrointestinal cancer, Gastrointestinal Carcinoid Tumor, Gastrointestinal Stromal Tumor, Germ cell tumor, Germinoma, Gestational choriocarcinoma, Gestational Trophoblastic Tumor, Giant cell tumor of bone, Glioblastoma multiforme, Glioma, Gliomatosis cerebri, Glomus tumor, Glucagonoma, Gonadoblastoma, Granulosa cell tumor, Hairy cell leukemia, Head and Neck Cancer, Heart cancer, Hemangioblastoma, Hemangiopericytoma, Hemangiosarcoma, Hematological malignancy, Hepatocellular carcinoma, Hepatosplenic T-cell lymphoma, Hereditary breast-ovarian cancer syndrome, Hodgkin's lymphoma, Hypopharyngeal Cancer, Hypothalamic Glioma, Inflammatory breast cancer, Intraocular Melanoma, Islet cell carcinoma, Islet Cell Tumor, Juvenile myelomonocytic leukemia, Kaposi's sarcoma, Kidney Cancer, Klatskin tumor, Krukenberg tumor, Laryngeal Cancer, Laryngeal cancer, Lentigo maligna melanoma, Leukemia, Lip and Oral Cavity Cancer, Liposarcoma, Lung cancer, Luteoma, Lymphangioma, Lymphangiosarcoma, Lymphoepithelioma, Lymphoid leukemia, Lymphoma, Macroglobulinemia, Malignant fibrous histiocytoma, Malignant Fibrous Histiocytoma of Bone, Malignant Glioma, Malignant Mesothelioma, Malignant peripheral nerve sheath tumor, Malignant rhabdoid tumor, Malignant triton tumor, MALT lymphoma, Mantle cell lymphoma, Mast cell leukemia, Mediastinal germ cell tumor, Mediastinal tumor, Medullary thyroid cancer, Medulloblastoma, Medulloepithelioma, Melanoma, Meningioma, Merkel Cell Carcinoma, Mesothelioma, Metastatic Squamous Neck Cancer with Occult Primary, Metastatic urothelial carcinoma, Mixed Mullerian tumor, Monocytic leukemia, Mouth Cancer, Mucinous tumor, Multiple Endocrine Neoplasia Syndrome, Multiple Myeloma, Mycosis fungoides, Myelodysplastic Syndromes, Myeloid leukemia, Myeloid sarcoma, Myeloproliferative Disease, Myxoma, Nasal Cavity Cancer, Nasopharyngeal Cancer, Neoplasm, Neurinoma, Neuroblastoma, Neurofibroma, Neuroma, Nodular melanoma, Non-Hodgkin Lymphoma, Non-melanoma Skin Cancer, Non-Small Cell Lung Cancer, Ocular oncology, Oligoastrocytoma, Oligodendroglioma, Oncocytoma, Optic nerve sheath meningioma, Oral Cancer, Oropharyngeal Cancer, Osteosarcoma, Ovarian Cancer, Ovarian Epithelial Cancer, Ovarian Germ Cell Tumor, Ovarian Low Malignant Potential Tumor, Paget's disease of the breast, Pancoast tumor, Pancreatic cancer, Papillary thyroid cancer, Papillomatosis, Paraganglioma, Paranasal Sinus Cancer, Parathyroid Cancer, Penile Cancer, Perivascular epithelioid cell tumor, Pharyngeal Cancer, Pheochromocytoma, Pineal Parenchymal Tumor of Intermediate Differentiation, Pineoblastoma, Pituicytoma, Pituitary adenoma, Pituitary tumor, Plasma Cell Neoplasm, Pleuropulmonary blastoma, Polyembryoma, Precursor T-lymphoblastic lymphoma, Primary central nervous system lymphoma, Primary effusion lymphoma, Primary Hepatocellular Cancer, Primary Liver Cancer, Primary peritoneal cancer, Primitive neuroectodermal tumor, Prostate cancer, Pseudomyxoma peritonei, Rectal Cancer, Renal cell carcinoma, Respiratory Tract Carcinoma Involving the NUT Gene on Chromosome 15, Retinoblastoma, Rhabdomyoma, Rhabdomyosarcoma, Richter's transformation, Sacrococcygeal teratoma, Salivary Gland Cancer, Sarcoma, Schwannomatosis, Sebaceous gland carcinoma, Secondary neoplasm, Seminoma, Serous tumor, Sertoli-Leydig cell tumor, Sex cord-stromal tumor, Sezary Syndrome, Signet ring cell carcinoma, Skin Cancer, Small blue round cell tumor, Small cell carcinoma, Small Cell Lung Cancer, Small cell lymphoma, Small intestine cancer, Soft tissue sarcoma, Somatostatinoma, Soot wart, Spinal Cord Tumor, Spinal tumor, Splenic marginal zone lymphoma, Squamous cell carcinoma, Stomach cancer, Superficial spreading melanoma, Supratentorial Primitive Neuroectodermal Tumor, Surface epithelial-stromal tumor, Synovial sarcoma, T-cell acute lymphoblastic leukemia, T-cell large granular lymphocyte leukemia, T cell leukemia, T-cell lymphoma, T-cell prolymphocytic leukemia, Teratoma, Terminal lymphatic cancer, Testicular cancer, Thecoma, Throat Cancer, Thymic Carcinoma, Thymoma, Thyroid cancer, Transitional Cell Cancer of Renal Pelvis and Ureter, Transitional cell carcinoma, Urachal cancer, Urethral cancer, Urogenital neoplasm, Uterine sarcoma, Uveal melanoma, Vaginal Cancer, Verner Morrison syndrome, Verrucous carcinoma, Visual Pathway Glioma, Vulvar Cancer, Waldenstrom's macroglobulinemia, Warthin's tumor, Wilms' tumor, or any combination thereof.

12. The method of any one of claims 1-11, wherein the cancer is liver cancer.

13. The method of claim 12, wherein the liver cancer is selected from hepatocellular carcinoma, hepatoma, cholangiocarcinoma, hepatoblastoma, hepatic carcinoma, hepatic angiosarcoma, and metastatic liver cancer.

14. The method of any one of claims 1-13, wherein the cancer is hepatocellular carcinoma, gastric cancer, or adenoid cystic carcinoma.

15. The method of any one of claims 1-14, wherein the rivoceranib or a pharmaceutically acceptable salt thereof is administered for 28 days or more.

16. The method of any one of claims 1-15, wherein the subject with mild or moderate hepatic impairment does not have a statistically significant difference in exposure of rivoceranib, compared to exposure in a subject with normal hepatic function who is administered the same amount of rivoceranib.

17. The method of claim 16, wherein the exposure is measured by Cₘₐₓ or AUC_{0-∞}.

18. A method of treating cancer in a subject with mild or moderate hepatic impairment, the method comprising:
assessing hepatic function of the subject;
determining that the subject has mild or moderate hepatic impairment; and
administering to the subject with mild or moderate hepatic impairment a therapeutically effective amount of rivoceranib or a pharmaceutically acceptable salt thereof that is equal to a therapeutically effective amount for a subject with normal hepatic function, wherein the therapeutically effective amount of rivoceranib or a pharmaceutically acceptable salt thereof is administered in a total daily amount of about 100 mg to about 700 mg.

19. The method of claim 18, wherein the pharmaceutically acceptable salt of rivoceranib comprises a rivoceranib mesylate salt.

20. The method of claim 18 or 19, wherein the total daily amount of rivoceranib or a pharmaceutically acceptable salt thereof is about 100 mg, about 200 mg, about 300 mg, about 400 mg, or about 500 mg.

21. The method of any one of claims 18-20, wherein the total daily amount of rivoceranib a pharmaceutically acceptable salt thereof is about 200 mg.

22. The method of any one of claims 18-21, wherein rivoceranib or a pharmaceutically acceptable salt thereof is administered orally.

23. The method of any one of claims 18-22, wherein rivoceranib or a pharmaceutically acceptable salt thereof is administered in tablet form.

24. The method of any one of claims 18-23, wherein rivoceranib or a pharmaceutically acceptable salt thereof is administered once daily.

25. The method of any one of claims 18-24, wherein the hepatic function is assessed using a Child-Pugh Scale.

26. The method of claim 25, wherein a subject with mild hepatic impairment has a Child-Pugh score of 5-6; a subject with moderate hepatic impairment has a Child-Pugh score of 7-9; and a subject with severe hepatic impairment has a Child-Pugh score of 10-15.

27. The method of any one of claims 18-26, wherein the subject has mild hepatic impairment.

28. The method of any one of claims 18-27, wherein the subject has moderate hepatic impairment.

29. The method of any one of claims 18-26, wherein the subject has a Child-Pugh Score of 5-6 or 7-9.

30. The method of any one of claims 18-29, wherein the cancer is selected from Acanthoma, Acinic cell carcinoma, Acoustic neuroma, Acral lentiginous melanoma, Acrospiroma, Acute eosinophilic leukemia, Acute lymphoblastic leukemia, Acute megakaryoblastic leukemia, Acute monocytic leukemia, Acute myeloblastic leukemia with maturation, Acute myeloid dendritic cell leukemia, Acute myeloid leukemia, Acute promyelocytic leukemia, Adamantinoma, Adenocarcinoma, Adenoid cystic carcinoma, Adenoma, Adenomatoid odontogenic tumor, Adrenocortical carcinoma, Adult T-cell leukemia, Aggressive NK-cell leukemia, AIDS-Related Cancers, AIDS-related lymphoma, Alveolar soft part sarcoma, Ameloblastic fibroma, Anal cancer, Anaplastic large cell lymphoma, Anaplastic thyroid cancer, Angioimmunoblastic T-cell lymphoma, Angiomyolipoma, Angiosarcoma, Appendix cancer, Astrocytoma, Atypical teratoid rhabdoid tumor, Basal cell carcinoma, Basal-like carcinoma, B-cell leukemia, B-cell lymphoma, Bellini duct carcinoma, Biliary tract cancer, Bladder cancer, Blastoma, Bone Cancer, Bone tumor, Brain Stem Glioma, Brain Tumor, Breast Cancer, Brenner tumor, Bronchial Tumor, Bronchioloalveolar carcinoma, Brown tumor, Burkitt's lymphoma, Cancer of Unknown Primary Site, Carcinoid Tumor, Carcinoma, Carcinoma in situ, Carcinoma of the penis, Carcinoma of Unknown Primary Site, Carcinosarcoma, Castleman's Disease, Central Nervous System Embryonal Tumor, Cerebellar Astrocytoma, Cerebral Astrocytoma, Cervical Cancer, Cholangiocarcinoma, Chondroma, Chondrosarcoma, Chordoma, Choriocarcinoma, Choroid plexus papilloma, Chronic Lymphocytic Leukemia, Chronic monocytic leukemia, Chronic myelogenous leukemia, Chronic Myeloproliferative Disorder, Chronic neutrophilic leukemia, Clear-cell tumor, Colon Cancer, Colorectal cancer, Craniopharyngioma, Cutaneous T-cell lymphoma, Degos disease, Dermatofibrosarcoma protuberans, Dermoid cyst, Desmoplastic small round cell tumor, Diffuse large B cell lymphoma, Dysembryoplastic neuroepithelial tumor, Embryonal carcinoma, Endodermal sinus tumor, Endometrial cancer, Endometrial Uterine Cancer, Endometrioid tumor, Enteropathy-associated T-cell lymphoma, Ependymoblastoma, Ependymoma, Epithelioid sarcoma, Erythroleukemia, Esophageal cancer, Esthesioneuroblastoma, Ewing's sarcoma, Extracranial Germ Cell Tumor, Extragonadal Germ Cell Tumor, Extrahepatic Bile Duct Cancer, Extramammary Paget's disease, Fallopian tube cancer, Fetus in fetu, Fibroma, Fibrosarcoma, Follicular lymphoma, Follicular thyroid cancer, Gallbladder Cancer, Ganglioglioma, Ganglioneuroma, Gastric Cancer, Gastric lymphoma, Gastrointestinal cancer, Gastrointestinal Carcinoid Tumor, Gastrointestinal Stromal Tumor, Germ cell tumor, Germinoma, Gestational choriocarcinoma, Gestational Trophoblastic Tumor, Giant cell tumor of bone, Glioblastoma multiforme, Glioma, Gliomatosis cerebri, Glomus tumor, Glucagonoma, Gonadoblastoma, Granulosa cell tumor, Hairy cell leukemia, Head and Neck Cancer, Heart cancer, Hemangioblastoma, Hemangiopericytoma, Hemangiosarcoma, Hematological malignancy, Hepatocellular carcinoma, Hepatosplenic T-cell lymphoma, Hereditary breast-ovarian cancer syndrome, Hodgkin's lymphoma, Hypopharyngeal Cancer, Hypothalamic Glioma, Inflammatory breast cancer, Intraocular Melanoma, Islet cell carcinoma, Islet Cell Tumor, Juvenile myelomonocytic leukemia, Kaposi's sarcoma, Kidney Cancer, Klatskin tumor, Krukenberg tumor, Laryngeal Cancer, Laryngeal cancer, Lentigo maligna melanoma, Leukemia, Lip and Oral Cavity Cancer, Liposarcoma, Lung cancer, Luteoma, Lymphangioma, Lymphangiosarcoma, Lymphoepithelioma, Lymphoid leukemia, Lymphoma, Macroglobulinemia, Malignant fibrous histiocytoma, Malignant Fibrous Histiocytoma of Bone, Malignant Glioma, Malignant Mesothelioma, Malignant peripheral nerve sheath tumor, Malignant rhabdoid tumor, Malignant triton tumor, MALT lymphoma, Mantle cell lymphoma, Mast cell leukemia, Mediastinal germ cell tumor, Mediastinal tumor, Medullary thyroid cancer, Medulloblastoma, Medulloepithelioma, Melanoma, Meningioma, Merkel Cell Carcinoma, Mesothelioma, Metastatic Squamous Neck Cancer with Occult Primary, Metastatic urothelial carcinoma, Mixed Mullerian tumor, Monocytic leukemia, Mouth Cancer, Mucinous tumor, Multiple Endocrine Neoplasia Syndrome, Multiple Myeloma, Mycosis fungoides, Myelodysplastic Syndromes, Myeloid leukemia, Myeloid sarcoma, Myeloproliferative Disease, Myxoma, Nasal Cavity Cancer, Nasopharyngeal Cancer, Neoplasm, Neurinoma, Neuroblastoma, Neurofibroma, Neuroma, Nodular melanoma, Non-Hodgkin Lymphoma, Non-melanoma Skin Cancer, Non-Small Cell Lung Cancer, Ocular oncology, Oligoastrocytoma, Oligodendroglioma, Oncocytoma, Optic nerve sheath meningioma, Oral Cancer, Oropharyngeal Cancer, Osteosarcoma, Ovarian Cancer, Ovarian Epithelial Cancer, Ovarian Germ Cell Tumor, Ovarian Low Malignant Potential Tumor, Paget's disease of the breast, Pancoast tumor, Pancreatic cancer, Papillary thyroid cancer, Papillomatosis, Paraganglioma, Paranasal Sinus Cancer, Parathyroid Cancer, Penile Cancer, Perivascular epithelioid cell tumor, Pharyngeal Cancer, Pheochromocytoma, Pineal Parenchymal Tumor of Intermediate Differentiation, Pineoblastoma, Pituicytoma, Pituitary adenoma, Pituitary tumor, Plasma Cell Neoplasm, Pleuropulmonary blastoma, Polyembryoma, Precursor T-lymphoblastic lymphoma, Primary central nervous system lymphoma, Primary effusion lymphoma, Primary Hepatocellular Cancer, Primary Liver Cancer, Primary peritoneal cancer, Primitive neuroectodermal tumor, Prostate cancer, Pseudomyxoma peritonei, Rectal Cancer, Renal cell carcinoma, Respiratory Tract Carcinoma Involving the NUT Gene on Chromosome 15, Retinoblastoma, Rhabdomyoma, Rhabdomyosarcoma, Richter's transformation, Sacrococcygeal teratoma, Salivary Gland Cancer, Sarcoma, Schwannomatosis, Sebaceous gland carcinoma, Secondary neoplasm, Seminoma, Serous tumor, Sertoli-Leydig cell tumor, Sex cord-stromal tumor, Sezary Syndrome, Signet ring cell carcinoma, Skin Cancer, Small blue round cell tumor, Small cell carcinoma, Small Cell Lung Cancer, Small cell lymphoma, Small intestine cancer, Soft tissue sarcoma, Somatostatinoma, Soot wart, Spinal Cord Tumor, Spinal tumor, Splenic marginal zone lymphoma, Squamous cell carcinoma, Stomach cancer, Superficial spreading melanoma, Supratentorial Primitive Neuroectodermal Tumor, Surface epithelial-stromal tumor, Synovial sarcoma, T-cell acute lymphoblastic leukemia, T-cell large granular lymphocyte leukemia, T cell leukemia, T-cell lymphoma, T-cell prolymphocytic leukemia, Teratoma, Terminal lymphatic cancer, Testicular cancer, Thecoma, Throat Cancer, Thymic Carcinoma, Thymoma, Thyroid cancer, Transitional Cell Cancer of Renal Pelvis and Ureter, Transitional cell carcinoma, Urachal cancer, Urethral cancer, Urogenital neoplasm, Uterine sarcoma, Uveal melanoma, Vaginal Cancer, Verner Morrison syndrome, Verrucous carcinoma, Visual Pathway Glioma, Vulvar Cancer, Waldenstrom's macroglobulinemia, Warthin's tumor, Wilms' tumor, or any combination thereof.

31. The method of any one of claims 18-30, wherein the cancer is liver cancer.

32. The method of claim 31, wherein the liver cancer is selected from hepatocellular carcinoma, hepatoma, cholangiocarcinoma, hepatoblastoma, hepatic carcinoma, hepatic angiosarcoma, and metastatic liver cancer.

33. The method of any one of claims 18-32, wherein the cancer is hepatocellular carcinoma, gastric cancer, or adenoid cystic carcinoma.

34. The method of any one of claims 18-33, wherein the rivoceranib or a pharmaceutically acceptable salt thereof is administered for 28 days or more.

35. The method of any one of claims 18-36, wherein a subject with mild or moderate hepatic impairment does not have a statistically significant difference in exposure of rivoceranib compared to exposure in a subject with normal hepatic function who is administered the same amount of rivoceranib.

36. The method of claim 35, wherein the exposure is measured by Cₘₐₓ or AUC_{0-∞}.

37. The method of any one of claims 18-36, further comprising discontinuing the administration of the rivoceranib or a pharmaceutically acceptable salt thereof when the patient no longer has cancer or when the patient has a Child-Pugh score of 10 or higher.

38. The method of claim 37, wherein if the subject is determined to have severe hepatic impairment, rivoceranib or a pharmaceutically acceptable salt thereof is not administered to that subject.

39. Rivoceranib or a pharmaceutically acceptable salt thereof for use in the treatment of cancer in a subject having mild or moderate hepatic impairment, wherein the rivoceranib or a pharmaceutically acceptable salt thereof is administered to the subject in a total daily in a range of about 100 mg to about 700 mg.

40. A dosing regimen for rivoceranib or a pharmaceutically acceptable salt thereof in the treatment of cancer in a subject having mild or moderate hepatic impairment, the dosing regimen comprising administering a total daily amount of rivoceranib or a pharmaceutically acceptable salt thereof in a range of about 100 mg to about 700 mg for at least one cycle, wherein each cycle is at least about 10 days.

41. The dosing regimen of claim 40, wherein each cycle is at least 28 days.
